SEQUENCE LISTING (2 sequences)

SEQ ID NO: 1 - protein, 257 aa, Artificial Sequence
  Amino acid sequence of anti-CD19 scFv
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGCGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSHHHHHH

SEQ ID NO: 2 - protein, 250 aa, Artificial Sequence
  Amino acid sequence of anti-CD3 scFv
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGCGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

(19) United States
(12) Patent Application Publication
(10) Pub. No.: US 2024/0181074 A1
(43) Pub. Date: Jun. 6, 2024

(54) PEGYLATED T CELL ENGAGER WITH DUAL SPECIFICITIES TO CD3 AND CD19

(71) Applicant: Shenzhen Enduring Biotech, Ltd., Shenzhen (CN)

(72) Inventors: Yu Wen, Shenzhen (CN); Shumin Liu, Shenzhen (CN); Shuangyu Tan, Shenzhen (CN); Dechun Wu, Shenzhen (CN)

(73) Assignee: Shenzhen Enduring Biotech, Ltd., Shenzhen (CN)

(21) Appl. No.: 18/551,136

(22) PCT Filed: Mar. 18, 2022
(86) PCT No.: PCT/CN2022/081598

§ 371 Date: Sep. 18, 2023

(30) Foreign Application Priority Data
Mar. 19, 2021 (WO) PCT/CN2021/081765

(51) Int. Cl. A61K 47/68; A61K 45/06; A61K 47/54; A61K 47/60; A61P 37/06

(52) U.S. Cl. CPC A61K 47/6849; A61K 45/06; A61K 47/545; A61K 47/60; A61P 37/06

(57) ABSTRACT

The provided is a T-BsAb (T-cell-engaging bispecific antibody) with dual affinities to CD3 on T cells and CD19 on B cells and use thereof in treating autoimmune diseases. In particular, the provided is a PEGylated T-BsAb with dual affinities to CD3 and CD19 and use thereof in treating multiple sclerosis (MS) as well as other autoimmune diseases.

This international patent application claims the benefit of international patent application No.: PCT/CN2021/081765 filed on Mar. 19, 2021, the entire content of which is incorporated by reference for all purpose.

### FIELD OF INVENTION

The present invention relates to a T-BsAb (T-cell-engaging bispecific antibody) with dual affinities to CD3 on T cells and CD19 on B cells and use thereof in treating autoimmune diseases. In particular, the invention relates to a PEGylated T-BsAb with dual affinities to CD3 and CD19 and use thereof in treating multiple sclerosis (MS) as well as other autoimmune diseases.

### BACKGROUND OF THE INVENTION

There are more than 80 different autoimmune diseases. Genetics, diet, infections, and exposure to chemicals might be involved (Campbell, A. W. 2014, Autoimmune Dis 2014, 152428). B cells play critical roles in the pathogenesis of autoimmune disease by secreting self-tissue damaging autoantibodies, presenting antigens, activating pro-inflammatory T cells and producing cytokines (Sabatino, J. J. et. al. 2019, Nat Rev Neurosci 20, 728-745; Lee, D. S. W. et. al. 2020, Nat Rev Drug Discov, 1-21). B cell depletion has been proven to be an effective therapeutic strategy for autoimmune disease (Hofmann, K. et. al. 2018, Front Immunol 9, 835). FDA approved B cell depletion therapies have been expanded to a multitude of autoimmune diseases ranging from organ specific diseases, like pemphigus and MS, to systemic diseases such as ANCA associated vasculitis, rheumatoid arthritis (RA) and systemic lupus erythematosus (SLE) (Barnas, J. L., et. al. 2019, Curr Opin Immunol 61, 92-99). These therapies are primarily monoclonal antibody based and target B cells with surface markers such as CD20 and CD19 (Townsend, M. J., et. al. 2010, Immunological Reviews 237, 264-283; Frampton, J. E. 2020, Drugs 80, 1259-1264). However, anti-CD20 antibodies (ocrelizumab, ofatumumab, Rituximab) cannot effectively remove autoantibody-secreting, long-lived plasma cells, which are extremely refractory to conventional immunosuppressive therapies (Chen, D. et al. 2016, The Journal of Immunology 196, 1541-1549). In fact, ˜20% of MS patients treated with rituximab experienced relapse of the disease at 48 weeks (Stephen L. Hauser, 2008, The New Engl and Journal of Medicine 358, 12). It is possible that long-lived autoreactive plasma cells that are not sufficiently eliminated by anti-CD20 antibody therapies could lead to the reactivation of the disease in a subset of MS patients. It has been reported that CD19⁺ B cells play more critical roles in pathogenesis of multiple sclerosis and anti-CD19 monoclonal antibodies show better therapeutic effects than anti-CD20 antibodies in treating the EAE (Experimental Autoimmune Encephalomyelitis) animal model of MS (Chen, D. et al. 2016, The Journal of Immunology 196, 1541-1549). However, anti-CD19 antibodies target and deplete almost all subsets of B cells, which could potentially pose a serious threat of virus infection to the patients. Recent studies showed that patients treated with B cell depletion agent experienced higher risks of virus infection and higher rate of severe disease and death (Loarce-Martos, J. et al. 2020, Rheumatol Int 40, 2015-2021).

Because not all CD19⁺ B cells are involved in autoimmune pathogenesis, targeting pathological specific B cells instead of depleting all CD19⁺ B cells is more efficient, promising and safer therapies to treat autoimmune disease such as MS.

Some investigators proposed that blinatumomab, a T-BsAb or BiTE (Bispecific T cell Engager) approved for the treatment of ALL (acute lymphoblastic leukemia) by redirecting T cells to CD19⁺ B cells for lysing the B cells, might be a candidate for the treatment of autoimmune diseases (Musette, P. et. al. 2018, Front Immunol 9, 622). However, clinical studies have shown frequent cytokine release syndrome (CRS) and central nerve system (CNS) toxicity in patients treated with blinatumomab. It has been suggested that the neurological toxicity of blinatumomab may result from the rapid release of inflammatory cytokines (Topp, M. S. et al. 2012, Blood 120, 670-670; Portell, C. A., et. al. 2013, Clin Pharmacol 5, 5-11; Bargou, R. et al. 2008, Science 321, 974-977; Topp, M. S. et al. 2011, J Clin Oncol 29 2493-2498; Topp, M. S. et al. 2012, Blood 120, 5185-5187). It is well expected that the safety requirements of drugs for treating chronic diseases are more stringent than those of oncological drugs. Since autoimmune diseases are chronic diseases, the frequent and severe CRS and CNS toxicity constitute a barrier for use of blinatumomab in the treatment of autoimmune diseases. Furthermore, blinatumomab is administered via continuous intravenous infusion through a portable mini pump owing to its 1.25 hour of short circulation half-life, which requires cancer patients to be hospitalized (a challenge for treatment of autoimmune disease patients) in addition to the high infection risk associated with long and continuous infusion (Portell, C. A., et. al. 2013, Clin Pharmacol 5, 5-11; Topp, M. S. et al. 2012, Blood 120, 5185-5187)¹².

Although blinatumomab had its huge success in the treatment of hematologic cancer, the above stated problems and challenges have motivated the development of T-BsAb with weaker T cell binding affinity to decouple the severe CRS toxicity with potent cytotoxicity, and to minimize cytokine release while still effectively activate cytotoxic T cells and form an immune synapse with the pathological target cells to kill the target cells.

### SUMMARY OF INVENTION

In one aspect, the invention provides a method of treating an autoimmune diseases, including:
administering an effective amount of a compound of Formula Ia, or a pharmaceutically acceptable salt thereof to the subject

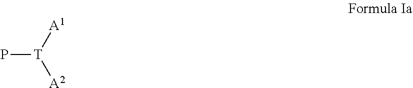

wherein
P is a non-immunogenic polymer;
T is a multifunctional (e.g. trifunctional) small molecule linker moiety and has one, two, or more functional groups that are capable of site-specific conjugation with one, two or more the same or different polypeptides;
Each of A¹ and A² is independently selected from an antibody, e.g. a single chain antibody (e.g. a single chain variable fragment, scFv), a single domain antibody (nanobody) or a Fab, in which one of A¹ and A² recognizes and binds to the antigen CD3 and the other recognizes and binds the antigen CD19. The antibody or Fab recognizing and binding to antigen CD19 may bind to the extracellular portion of CD19. The antibody or Fab recognizing and binding to antigen CD3 may bind any one of the CD3 complex subunits in the T cell receptor complex, namely CD3 gamma, CD3 delta, CD3 epsilon, CD3 zeta, and CD3 eta (Kuhns, M. S., et. al. 2006, Immunity 24, 133-139; 2008, Risueno, R. M., PLoS One 3, e1747).

Another aspect of the invention provides a method of treating an autoimmune disease including administrating an effective amount of a compound of Formula Ib, or a pharmaceutically acceptable salt thereof to the subject:

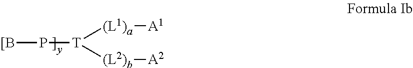

wherein:
P is a non-immunogenic polymer;
B is H or a terminal capping group selected from C₁₋₂₀ alkyl and aryl, e.g. C₁₋₁₀ alkyl and aryl, wherein one or more carbons of said alkyl or aryl are optionally replaced with a heteroatom;
T is a tri-functional (e.g. an amino acid) linker having one, two, or more functional groups that, after derivatization and/or extension with a bifunctional spacer, are capable of site-specific conjugation with A¹ and A² or their derivatives, wherein the linkage between T and (L¹)ₐ and the linkage between T and (L²)_{b} can be same or different;
each of L¹ and L² is independently a bifunctional linker (e.g. a peptide);
each of a and b is an integer selected from 1, 2, 3, 4, 5, 6, 7, 8, 9, and 10;
each of A¹ and A² is independently selected from an antibody e.g. a single chain antibody (e.g. a single chain variable fragment, scFv), a single domain antibody (nanobody) or a Fab, in which one of A¹ and A² recognizes and binds to the antigen CD3 and the other recognizes and binds the antigen CD19;
and
y is an integer selected from 1-10, e.g. an integer selected from 1, 2, 3, 4, 5, 6, 7, 8, 9, and 10.

In one aspect, the antibody or Fab recognizing and binding to antigen CD19 may bind to the extracellular portion of CD19. In some embodiment, the antibody recognizing and binding to antigen CD19 is an anti-CD19 scFv.

In some embodiment, the anti-CD19 scFv has following amino acid sequence:

(SEQ ID NO: 1)
DIQLTQSPASLAVSLGQRATISCKASQSVDYDGDSYLNWYQQIPGQPPK
LLIYDASNLVSGIPPRFSGSGSGTDFTLNIHPVEKVDAATYHCQQSTED
PWTFGGGTKLEIKGCGGGSGGGGSGGGGSQVQLQQSGAELVRPGSSVKI
SCKASGYAFSSYWMNWVKQRPGQGLEWIGQIWPGDGDTNYNGKFKGKAT
LTADESSSTAYMQLSSLASEDSAVYFCARRETTTVGRYYYAMDYWGQGT
TVTVSSHHHHHH

In another aspect of the invention, the antibody or Fab recognizing and binding to antigen CD3 may bind any one of the CD3 complex subunits in the T cell receptor complex, namely CD3 gamma, CD3 delta, CD3 epsilon, and CD3 zeta eta. In some embodiment, the antibody recognizing and binding to antigen CD3 is an anti-CD3 scFv.

In some embodiments, the anti-CD3 scFv has the following amino acid sequence:

(SEQ ID NO: 2)
DIKLQQSGAELARPGASVKMSCKTSGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSVEGCGSGGSGGSGGSGGVDDIQLTQSPA
IMSASPGEKVTMTCRASSSVSYMNWYQQKSGTSPKRWIYDTSKVASGVP
YRFSGSGSGTSYSLTISSMEAEDAATYYCQQWSSNPLTFGAGTKLELKH
HHHHH

The non-immunogenic polymer can be selected from the group consisting of polyethylene glycol (PEG), dextrans, carbohydrate-base polymers, polyalkylene oxide, polyvinyl alcohols, hydroxypropyl-methacrylamide (HPMA), and a co-polymer thereof. Preferably, the non-immunogenic polymer is PEG, such as a branched PEG or a linear PEG. In some embodiments, at least one terminal of the linear PEG or branch PEG is capped with H, methyl or low molecule weight alkyl group. The total molecule weight of the PEG can be 3,000 to 100,000 Daltons, e.g., 5,000 to 80,000, 10,000 to 60,000, or 20,000 to 40,000 Daltons. The PEG can be linked to the tri-functional linker T moiety either through a permanent bond or a cleavable bond.

The functional groups (e.g., the functional groups for site-specific conjugation) that form linkages within (L¹)ₐ or (L²)_{b}, between (L¹)ₐ and protein A¹, between (L²)_{b} and protein A², between T and L¹ or between T and L² can be selected from the group consisting of alkyl halide, acid halide, aldehyde, ketone, ester, anhydride, carboxylic acid, amide, amine, hydrazide, alkylhydrazines, hydroxy, epoxide, thiol, maleimide, 2-pyridyldithio variant, aromatic sulfone or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, triarylphosphine, and the like.

In some embodiments, each of (L¹)ₐ and (L²)_{b} can independently comprise a linkage formed from azide and alkyne or formed from maleimide and thiol. In other embodiments, each of (L¹)ₐ, (L²)_{b} and T can independently be an amino acid or a peptide having 2-50 amino acid units. In some examples, the alkyne can be dibenzocyclooctyl (DBCO).

In some other embodiments, T is lysine, P is PEG, and y is 1, while the alkyne is dibenzocyclooctyl (DBCO).

In some embodiments, one of A¹ and A² can be derived from an azide tagged antibody, antibody chain, antibody fragment, single chain antibody or single domain antibody, wherein the azide is conjugated to an alkyne in the respective (L¹)ₐ or (L²)_{b}; the other of A¹ and A² can be derived from a thiol tagged antibody, antibody chain, antibody fragment, single chain antibody or single domain antibody, wherein the thiol is conjugated to a maleimide in the respective (L¹)ₐ or (L²)_{b}.

The above-described molecule or compound can be made according to a method comprising: (i) preparing a non-immunogenic polymer with terminal bi-functional groups capable of site-specific conjugation with two different polypeptide, e.g. two different antibodies or modified forms thereof; and (ii) stepwise site-specific conjugating the non-immunogenic polymer with an anti-CD3 antibody (or an antigen-binding fragment thereof) and an anti-CD19 antibody (or an antigen-binding fragment thereof) or their modified forms to form a compound of Formula Ia or Ib.

Alternatively, the above-described PEGylated T-BsAb molecule or compound can be made according to a method comprising: preparing an anti-CD3 and anti-CD19 fusion protein with a thiol tag, followed by PEGylation of the fusion protein with a thiol specific PEG reagent such as PEG maleimide.

The autoimmune diseases to be treated with the methods and compounds described herein include pemphigus, neuromyelitis optica/neuromyelitis optica-spectrum disorders (NOD/NMOD), multiple sclerosis (MS), ANCA associated vasculitis, rheumatoid arthritis (RA), Crohn's disease, inflammatory bowel disease (IBD) and systemic lupus erythematosus (SLE), asthma, psoriasis/psoriatic arthritis, addison's disease, graves' disease, atopic dermatitis, erythematosus, type 1 diabetes and others. The forgoing list is not meant to be exclusive and those of ordinary skill will realize that other autoimmune diseases not specifically mentioned herein are intended for inclusion.

In some embodiments, the autoimmune disease to be treated is MS. In some embodiments, the disease is MS showing resistance or refractory to conventional immunosuppressive therapies with the compounds such as Tecfidera, Gilenya, Tysabri, Aubagio, Mavenclad, Copaxone, IFN-β-1a, IFN-β-1b, anti-CD52 antibody (Alemtuzumab, Alemtuzumab), Natalizumab, anti-CD19 agents (Inebilizumab, Obexelimab), or anti-CD20 agents (Rituximab, ocrelizumab, ofatumumab).

In some embodiments, the disease to be treated is an autoimmune disease showing resistance or refractory to therapies associated with administration of B cell depletion agents such as anti-CD19 or anti-CD20 monoclonal antibodies. In other embodiments, the disease to be treated is an autoimmune disease showing resistance or refractory to therapies associated with administration of anti-CD3×anti-CD20 bispecific antibodies.

In some embodiments, the compound of Formula Ia or Ib, or a pharmaceutically acceptable salt thereof is administrated to the subject at the onset of treatment (e.g. as the first-line therapy) or in subsequent rounds of treatment (e.g. as the second-line, third line, or fourth-line therapies) of the auto-immune disease. In some embodiments, the auto-immune disease is MS. In some embodiments, the compound is effective for the treatment of MS relapsed after discontinuation of the treatment.

In some embodiments, the compound of Formula Ta or Tb, or a pharmaceutically acceptable salt thereof is administrated in an amount of from 0.05 mg/kg/dose to 50 mg/kg/dose. In some embodiments, the compound described herein is administrated once to eight times every 4-8 weeks for each treatment cycle, or once to four times in 4-8 weeks, followed by one week rest period for each cycle until desired results are demonstrated.

In some embodiments, an effective amount of the compound is administrated concurrently or sequentially with another therapeutic agent for treating the autoimmune disease.

An advantage of the present method and compound is that the PEGylated anti-CD3×anti-CD19 T-BsAbs described herein have reduced-toxicity and/or overcome problems encountered by prior pharmaceutical agents.

In another aspect, the present invention provides a composition, e.g. a pharmaceutical composition, wherein the composition comprises the compound of Formula Ia or Ib, or a pharmaceutically acceptable salt thereof, and optionally a pharmaceutically acceptable carrier, excipient, or a diluent.

In some embodiments, the composition further comprises a second therapeutic agent for treating the auto-immune disease. In some embodiments, the second therapeutic agent can be selected from small molecular agents such as Tecfidera, Gilenya, Tysabri, Aubagio, Mavenclad; peptide agent such as Copaxone, protein biologics such as IFN-β-1a, IFN-β-1b, anti-CD52 antibodies (Alemtuzumab, Alemtuzumab), Natalizumab; B cell depletion agents such as anti-CD19 agents (Inebilizumab, Obexelimab in Phase II), anti-CD20 agents (Rituximab, ocrelizumab, ofatumumab).

In one aspect, the present invention provides use of the compound of Formula Ta, Tb, or a pharmaceutically acceptable salt thereof or the composition described above in the manufacture of a medicament for preventing, treating or lessening an autoimmune disease (e.g. MS) in a subject.

In another aspect, the present invention provides the compound of Formula Ta or Tb, or a pharmaceutically acceptable salt thereof or the composition described above, for use in preventing, treating or lessening an autoimmune disease (e.g. MS) in a subject.

The details of one or more embodiments of the invention are set forth in the following description and drawing. Other features, objectives, and advantages of the invention will be apparent from the descriptions and from the claims.

### BRIEF DESCRIPTION OF THE DRAWINGS

FIG. 1: ELISA assay comparing binding affinity to human CD19 or human CD3E&CD3D protein in Example 1.

FIG. 2: No drug-specific cytotoxicity to CD19⁻ cells was observed for JY108 in Example 2.

FIG. 3: Elimination of B cells in PBMC by JY108 in Example 2.

FIG. 4: E:T ratio effect on JY108 efficacies in Example 2.

FIG. 5: Cell lines effect on JY108 efficacies in Example 2.

FIG. 6: In vivo efficacy of JY108 to pre-B ALL tumor model in Example 3.

FIG. 7: Pharmacokinetic property of JY108 in Example 4.

FIG. 8: Binding of JY108 to targets in transgenic animal model in Example 5

FIG. 9: Spleen cells from transgenic mice (hCD3hCD19) as effectors for JY108 in Example 5.

FIG. 10: No acute toxicity of JY108 at single dose of 10 mg/kg in Example 6.

FIG. 11: Less cytokine release from human whole blood by JY108 in Example 7.

FIG. 12: JY108 significantly ameliorated EAE symptoms in Example 8.

FIG. 13: LFB staining of histopathological sections in Example 8.

FIG. 14: MOG specific autoantibody depletion by JY108 in Example 8.

FIG. 15: Head-to-head comparison of JY108 to MEDI-551 in Example 9.

FIG. 16: Partial removal of CD19⁺ B cell by JY108 in Example 10.

FIG. 17: B_{reg} cells are resistant to both MEDI-551 and JY108 in Example 10.

### DETAILED DESCRIPTION OF THE INVENTION

In this invention, a method of treating autoimmune diseases, in particularly MS, using PEGylated T-BsAbs with dual affinities to human CD3 of T cells and human CD19 of B cells is provided. The PEGylated T-BsAbs are capable of selective depletion of pathogenic CD19 expressing B cells with minimal cytokine release.

### A. Method of Treatment

In one aspect, this invention provides a method of treating an autoimmune disease, comprising:
administering an effective amount of a compound of Formula Ia, or a pharmaceutically acceptable salt thereof to the subject,

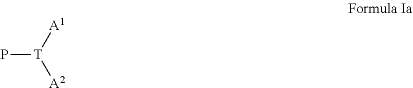

wherein
P is a non-immunogenic polymer;
T is a multifunctional (e.g. trifunctional) small molecule linker moiety and has one, two, or more functional groups that are capable of site-specific conjugation with one, two or more the same or different polypeptides;
Each of A¹ and A² is independently selected from an antibody, e.g. a single chain antibody (e.g. a single chain variable fragment, scFv), a single domain antibody (nanobody) or a Fab, in which one of A¹ and A² recognizes and binds to the antigen CD3 and the other recognizes and binds the antigen CD19. The antibody or Fab recognizing and binding to antigen CD19 may bind to the extracellular portion of CD19. The antibody or Fab recognizing and binding to CD3 may bind any one of the CD3 complex subunits in the T cell receptor complex, namely CD3 gamma, CD3 delta, CD3 epsilon, and CD3 zeta eta.

Another aspect of the invention provides a method of treating an autoimmune disease including administrating an effective amount of a compound of Formula Ib, or a pharmaceutically acceptable salt thereof to the subject:

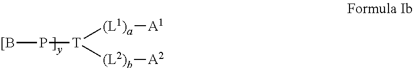

wherein:
P is a non-immunogenic polymer;
B is H or a terminal capping group selected from C₁₋₂₀ alkyl and aryl, e.g. C₁₋₁₀ alkyl and aryl, wherein one or more carbons of said alkyl or aryl are optionally replaced with a heteroatom;
T is a tri-functional (e.g. an amino acid) linker having one, two, or more functional groups that, after derivatization and/or extension with a bifunctional spacer, are capable of site-specific conjugation with A¹ and A² or their derivatives, wherein the linkage between T and (L¹)ₐ and the linkage between T and (L²)_{b} can be same or different;
each of L¹ and L² is independently a bifunctional linker (e.g. a peptide);
each of a and b is an integer selected from 1, 2, 3, 4, 5, 6, 7, 8, 9, and 10;
each of A¹ and A² is independently selected from an antibody e.g. a single chain antibody (e.g. a single chain variable fragment, scFv), a single domain antibody (nanobody) or a Fab, in which one of A¹ and A² recognizes and binds to the antigen CD3 and the other recognizes and binds the antigen CD19; and
y is an integer selected from 1, 2, 3, 4, 5, 6, 7, 8, 9, and 10.

In one aspect, the antibody or Fab recognizing and binding to antigen CD19 may bind to the extracellular portion of CD19. In some embodiment, the antibody recognizing and binding to antigen CD19 is an anti-CD19 scFv.

In some embodiment, the anti-CD19 scFv has following amino acid sequence:

(SEQ ID NO: 1)
DIQLTQSPASLAVSLGQRATISCKASQSVDYDGDSYLNWYQQIPGQPPK
LLIYDASNLVSGIPPRFSGSGSGTDFTLNIHPVEKVDAATYHCQQSTED
PWTFGGGTKLEIKGCGGGSGGGGSGGGGSQVQLQQSGAELVRPGSSVKI
SCKASGYAFSSYWMNWVKQRPGQGLEWIGQIWPGDGDTNYNGKFKGKAT
LTADESSSTAYMQLSSLASEDSAVYFCARRETTTVGRYYYAMDYWGQGT
TVTVSSHHHHHH

In another aspect of the invention, the antibody or Fab recognizing and binding to antigen CD3 may bind any one of the CD3 complex subunits in the T cell receptor complex, namely CD3 gamma, CD3 delta, CD3 epsilon, and CD3 zeta eta. In some embodiment, the antibody recognizing and binding to antigen CD3 is an anti-CD3 scFv.

In some embodiments, the anti-CD3 scFv has the following amino acid sequence:

(SEQ ID NO: 2)
DIKLQQSGAELARPGASVKMSCKTSGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSVEGCGSGGSGGSGGSGGVDDIQLTQSPA
IMSASPGEKVTMTCRASSSVSYMNWYQQKSGTSPKRWIYDTSKVASGVP
YRFSGSGSGTSYSLTISSMEAEDAATYYCQQWSSNPLTFGAGTKLELKH
HHHHH

### B. Polymer Moiety P

The non-immunogenic polymer P in the compound can be selected from the group consisting of polyethylene glycol (PEG), dextrans, carbohydrate-base polymers, polyalkylene oxide, polyvinyl alcohols, hydroxypropyl-methacrylamide (HPMA), and a co-polymer thereof. Preferably, the non-immunogenic polymer is PEG, such as a branched PEG or a linear PEG. In some embodiments, at least one terminal of the linear PEG or branch PEG is capped with H, methyl or low molecule weight alkyl group. The total molecule weight of the PEG can be 3,000 to 100,000 Daltons, e.g., 5,000 to 80,000, 10,000 to 60,000, or 20,000 to 40,000 Daltons. The PEG can be linked to the tri-functional linker T moiety either through a permanent bond or a cleavable bond.

The polymer may comprise a terminal group capable of being functionalized, activated, or conjugated to a reaction partner. Non-limiting examples of the terminal group include hydroxyl, amino, carboxyl, thiol, and halide.

In some embodiments, y is 1 and Formula Ib represents a compound with a pendent polymer chain. The terminal B may serve as a capping group.

In some embodiments, y is 2, 3, 4, 5 or 6 and Formula Ib represents a compound comprising a branched polymer moiety. In some embodiments, the B in [B—P]y is a low molecular weight C₁₋₁₀ alkyl group such as methyl, ethyl, and butyl, wherein one or more of the carbons may be replaced by a heteroatom (e.g. O, S, and N).

In another embodiment of present invention, an alternative branched PEG can be used.

The branched P moiety can be derived from a compound of the formula:

(B-PEG)ₘL-Sᵢ—Fᵢ
wherein:
PEG is polyethylene glycol. m is an integer greater than 1 to preferably provide a polymer having a total molecule weight of from 3000 to 50000 Daltons or greater if desired. B is methyl or other low molecule weight alkyl group. L is a functional linkage moiety to that two or more PEGs are attached. Examples of such linkage moiety include: any amino acid such as glycine, alanine, lysine, or 1,3-diamino-2-propanol, triethanolamine, any 5 or 6 member aromatic ring or aliphatic ring with more than two functional groups attached. S is any non-cleavable spacer. F is a terminal functional group such as hydroxyl, carboxyl, thiol, amino group, and the like. i is 0 or 1.

When i equals to 0, the formula is:

(B-PEG)ₘL
wherein PEG, m, B and L have the same definitions described above.

### C. Trifunctional Linker T

T represents a trifunctional linker, connecting with P, (L¹)ₐ and (L²)_{b}. T may be derived from molecules with any combination of three functional groups, non-limiting examples of which include hydroxyl, amino, hydrazinyl, carboxyl, thiol, and halide. The functional groups in the trifunctional linker may be the same or different. One or more of the functional groups of the trifunctional linker may be converted into one or more other groups before or after the reaction between T and the reaction partners. For example, a hydroxyl group may be converted into a mesylate or a tosylate group. A halide may be displaced with an azido group. An acid functional group of T may be converted to an alkyne function group by coupling with an amino group bearing a terminal alkyne.

In some embodiments, T may be derived from a natural or unnatural amino acid selected from the group consisting of cysteine, lysine, asparagine, aspartic, glutamic acid, glutamine, histidine, serine, threonine, tryptophan, tyrosine or genetically-encoded alkene lysine (such as N6-(hex-5-enoyl)-L-lysine), 2-Amino-8-oxononanoic acid, m- or p-acetyl-phenylalanine, amino acid bearing a β-diketone side chain (such as 2-amino-3-(4-(3-oxobutanoyl)phenyl)propanoic acid), (S)-2-amino-6-(((1R,2R)-2-azidocyclopentyloxy)carbonylamino) hexanoic acid, azidohomoalanine, pyrrolysine analogue N⁶-((prop-2-yn-1-yloxy)carbonyl)-L-lysine, (S)-2-Amino-6-pent-4-ynamidohexanoic acid, (S)-2-Amino-6-((prop-2-ynyloxy)carbonylamino)hexanoic acid, (S)-2-Amino-6-((2-azidoethoxy)carbonylamino)hexanoic acid, p-azidophenylalanine, para-azidophenylalanine, N^{ε}-Acryloyl-1-lysine, Nε-5-norbornene-2-yloxycarbonyl-1-lysine, N-ε-(Cyclooct-2-yn-1-yloxy)carbonyl)-L-lysine, N-ε-(2-(Cyclooct-2-yn-1-yloxy)ethyl) carbonyl-L-lysine, and genetically encoded tetrazine amino acid (such as 4-(6-methyl-s-tetrazin-3-yl)aminophenylalanine).

### D. Bifunctional Linker L¹ and L²

Each of the linker moieties L¹ and L² comprises a linker chain, internal linkages and/or a terminal linkages. The linker chain may be independently selected from an amino acid or a peptide having 2 to 50 amino acid residues, or —(CH₂)ₘXY(CH₂)ₙ—, —X(CH₂)ₘO(CH₂CH₂O)ₚ(CH₂)ₙY—, —(CH₂)ₘX—Y(CH₂)ₙ, —(CH₂)ₘheterocyclyl-, —(CH₂)ₘX—, —X(CH₂)ₘY—, wherein m, n, and p in each instance are independently an integer ranging from 0 to 25; X and Y in each instance are independently selected from the group consisting of C(═O), CR₁R₂, NR₃, S, O, or Null, wherein R₁ and R₂ independently represent hydrogen, C₁₋₁₀ alkyl or (CH₂)₁₋₁₀C(═O), R₃ is H or a C₁₋₁₀ alkyl, and wherein the heterocyclyl is derived from an maleimido, strained alkenes and alkynes, azide or a tetrazolyl moiety.

In one aspect, the linker chain may be independently selected from: —(CH₂)ₐC(O)NR¹(CH₂)_{b}—, —(CH₂)ₐO(CH₂CH₂O)_{c}—, —(CH₂)ₐheterocyclyl-, —(CH₂)ₐC(O)—, —(CH₂)ₐNR¹—, —CR¹═N—NR¹—, —CR¹═N—O—, —CR¹═N—NR²—CO—, —N═N—CO—, —S—S—, wherein a, b, and c are each an integer independently selected from 0 to 25 with all subunits included; and R¹ and R² independently represent hydrogen or a C1-C10 alkyl.

Heterocyclyl linkage group of L¹ and L² (at an internal position or at a terminal position) may be derived from a maleimido-based moiety. Non-limiting examples of suitable precursors include N-succinimidyl 4-(maleimidomethyl) cyclohexanecarboxylate (SMCC), N-succinimidyl-4-(N-maleimidomethyl)-cyclohexane-1-carboxy-(6-amidocaproate) (LC-SMCC), κ-maleimidoundecanoic acid N-succinimidyl ester (KMUA), γ-maleimidobutyric acid N-succinimidyl ester (GMBS), ε-maleimidcaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI).

Alternatively, the heterocyclyl linkage group of L¹ and L² may be tetrazolyl, trans-cyclooctene, azide or strained alkyne. The heterocyclyl triazolyl linkages, for example, may be formed from conjugations of two different linker moieties: azide and strained alkyne. Thus, the heterocyclyl group may also serve as a linkage point.

In some embodiments, (L¹)ₐ and/or (L²)_{b} comprises:
X¹—(CH₂)ₐC(O)NR¹(CH₂)_{b}O(CH₂CH₂O)_{c}(CH₂)ₐC(O)—, or
X³—(CH₂)ₐC(O)NR¹(CH₂)_{b}O(CH₂CH₂O)_{c}(CH₂)ₐ X² (CH₂)eN R²,
wherein X¹, X² and X³ may be the same or different and independently represent a heterocyclyl group;
a, b, c, d and e are each an integer independently selected from 1-25; and
R¹ and R² independently represent hydrogen or a C1-C10 alkyl.

In some embodiments, X¹ and/or X³ is derived from a maleimido-based moiety. In some embodiments, X² represents a triazolyl group. In some embodiments, R¹ and R² each represent a hydrogen. In some embodiments, a, b, c, d and e are each an integer independently selected from 0, 1, 2, 3, 4, 5, 6, 7, 8, 9, and 10.

In some other non-limiting exemplary embodiments, each of the linker moieties L¹ and L² may also be derived from a haloacetyl-based moiety selected from N-succinimidyl-4-(iodoacetyl)-aminobenzoate (SIAB), N-succinimidyl iodoacetate (SIA), N-succinimidyl bromoacetate (SBA), and N-succinimidyl 3-(bromoacetamido)propionate (SBAP).

### E. Linkage Group

Different moieties of the compounds or conjugates of the present invention may be connected via various chemical linkages. Examples include but are not limited to amide, ester, disulfide, ether, amino, carbamate, hydrazine, thioether, and carbonate.

For instance, the terminal hydroxyl group of a PEG moiety (P) may be activated and then coupled with lysine (T) to provide a desirable linkage point between P and T of Formula Ia or Ib. Meanwhile, the linkage group between T and L¹ or L² may be an amide resulting from the reaction of the amino group of a linker L¹ or L² with the carboxyl group of Lysine (T). Alternatively, the linkage group between T and L¹ or L² may be an amide resulting from the reaction of the amino group of T with activated carboxyl group of a linker L¹ or L². Depending on the desirable characteristics of the compound or conjugate, suitable linkage groups may also be incorporated between the antibody moiety (A) and the adjacent linker (L¹ or L²) and between or within individual linkers of L¹ or L².

In some embodiments, the linkage group between different moieties of the compounds or conjugates may be derived from coupling of a pair of functional groups which bear inherent chemical affinity and selectivity for each other. These types of coupling or ring formation allow for site-specific conjugation for the introduction of a particular polypeptide or antibody moiety. Non-limiting examples of these functional groups that lead to site-specific conjugation include thiol, maleimide, 2′-pyridyldithio variant, aromatic or vinyl sulfone, acrylate, bromo or iodo acetamide, azide, alkyne, dibenzocyclooctyl (DBCO), carbonyl, 2-amino-benzaldehyde or 2-amino-acetophenone group, hydrazide, oxime, potassium acyltrifluoroborate, O-carbamoylhydroxylamine, trans-cyclooctene, tetrazine, and triarylphosphine.

In some embodiments, each of (L¹)ₐ or (L²)_{b} can independently comprise a linkage formed from azide and alkyne or formed from maleimide and thiol. In other embodiments, each of (L¹)ₐ, (L²)_{b} and T can be an amino acid or a peptide having 2-50 amino acid units.

In some embodiments, the alkyne can be dibenzocyclooctyl (DBCO). In others, T is lysine, P is PEG, and y is 1, while the alkyne is dibenzocyclooctyl (DBCO). In another embodiments, one of A¹ and A² can be derived from an azide tagged antibody, antibody chain, antibody fragment, single chain antibody or single domain antibody, wherein the azide is conjugated to an alkyne in the respective (L¹)ₐ or (L²)_{b}; the other of A¹ and A² can be derived from a thiol tagged antibody, antibody chain, antibody fragment, single chain antibody or single domain antibody, wherein the thiol is conjugated to a maleimide in the respective (L¹)ₐ or (L²)_{b}.

### F. Synthesis of PEGylated T-BsAb with Dual Specificities to CD3 and CD19

In some embodiments, P represents a PEG moiety. In some exemplary embodiments, a terminal functional group of PEG such as hydroxyl or carboxyl group or the like, is activated and conjugated with a trifunctional small molecule moiety such as Boc protected lysine to form a terminal branched heterobifunctional PEG. The newly formed carboxyl group is then converted to alkyne group by coupling with a small molecule spacer that has alkyne group. The naked amino group after Boc deprotection is conjugated with another small molecule spacer that has maleimide group to form a terminal branched maleimide/alkyne heterobifunctional PEG. The resulting maleimide/alkyne terminal branched heterobifunctional PEG is site-specifically conjugated with a thiol tagged single chain anti-CD3 antibody and an azide tagged single chain anti-CD19 antibody consecutively to form a PEGylated T-BsAb.

Details of the synthesis are described in patent application WO 2018/075308A1 entitled “Long Acting Multi-Specific Molecules and Related Methods”, the contents of which are incorporated herein by reference in its entirety.

### G. Compositions/Formulations

The present invention also provides a composition, e.g., a pharmaceutical composition, containing PEGylated T-BsAb molecules of the present invention, optionally formulated together with a pharmaceutically acceptable carrier. For example, a pharmaceutical composition of the invention may comprise a PEGylated T-BsAb molecule that binds to both CD3 and CD19.

Therapeutic formulations of this invention may be prepared by mixing the PEGylated T-BsAb having the desired degree of purity with optional physiologically acceptable carriers, excipients or stabilizers (Remington's Pharmaceutical Sciences 1980, 16th edition, Osol, A. Ed.), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyl dimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS, or polyethylene glycol (PEG).

The formulation may also contain more than one active compound as necessary for the particular indication to be treated, preferably those with complementary activities that do not adversely affect each other. For instance, the formulation may further comprise another antibody, or an anti-autoimmune-disease agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 1980, 16th edition, Osol, A. Ed. Sustained-release preparations may be prepared. Suitable examples of sustained-release preparations include semipermeable matrices of solid hydrophobic polymers containing the PEGylated T-BsAb molecules, which matrices are in the form of shaped articles, e.g., films, or microcapsule. Examples of sustained-releasable matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies may be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Pharmaceutical compositions of the invention may be administered in combinational therapies, i.e., combined with other agents. Examples of therapeutic agents that may be used in combinational therapies include: small molecule agents such as Tecfidera, Gilenya, Tysabri, Aubagio, Mavenclad; peptide agents such as Copaxone, recombinant polypeptides or proteins such as IFN-β-1a, IFN-β-1b, anti-CD52 antibodies (Alemtuzumab, Alemtuzumab), Natalizumab; B cell depletion agents such as anti-CD19 agents (Inebilizumab, Obexelimab in Phase II), anti-CD20 agents (Rituximab, ocrelizumab, ofatumumab and the like.

The formulations to be used for in vivo administration should be sterile. This may be readily accomplished by filtration through sterile filtration membranes. Sterile injectable solutions may be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

### H. Dosages

The amount of active ingredient which may be combined with a carrier material to produce a single dosage form will vary depending upon the subject to be treated, and the particular mode of administration. The amount of active ingredient which may be combined with a carrier material to produce a single dosage form will generally be that amount of the composition which produces a therapeutic effect. Generally, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. Specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of PEGylated T-BsAb of this invention, the dosage ranges from about 0.0001 to 100 mg/kg, and more usually 0.05 to 50 mg/kg of the host body weight. For example dosages may be 0.01 mg/kg body weight, 0.1 mg/kg body weight, 1 mg/kg body weight, 5 mg/kg body weight, 10 mg/kg body weight or 50 mg/kg body weight or within the range of 0.1-10 mg/kg. An exemplary treatment regime entails administration twice or three times per week, once per week, once every two weeks, once every three weeks, once every four weeks, once a month, once every 3 months or once every three to 6 months. Preferred dosage regimens for PEGylated T-BsAb molecules of the invention include 0.25 mg/kg body weight or 1 mg/kg body weight via intravenous administration, with the PEGylated T-BsAb molecule being given using one of the following dosing schedules: (i) every four weeks for eight dosages, then every three months; (ii) every two weeks; (iii) 1 mg/kg body weight once followed by 0.25 mg/kg body weight every two weeks.

Alternatively, PEGylated T-BsAb molecules may be administered as a sustained release formulation, in which case less frequent administration is required. Dosage and frequency vary depending on the half-life of the PEGylated T-BsAb molecules in the patient. In general, T-BsAbs modified with higher molecular weight PEG have longer half-life. The dosage and frequency of administration may vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient may be administered a prophylactic regime. Actual dosage levels of the active ingredients in the pharmaceutical compositions of the present invention may be varied so as to obtain an amount of the active ingredient which is effective to achieve the desired therapeutic response for a particular patient, composition, and mode of administration. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of a PEGylated T-BsAb molecule of the invention preferably results in a decrease in severity of disease symptoms, an increase in frequency and duration of disease symptom-free periods, or a prevention of impairment or disability due to the disease affliction. For example, for the treatment of MS, a “therapeutically effective dosage” preferably lower the clinical score by at least about 20%, more preferably by at least about 40%, even more preferably by at least about 60%, and still more preferably by at least about 80% relative to untreated subjects. The ability of an agent or compound to lower clinical score may be evaluated in an animal model system predictive of efficacy in human diseases. Alternatively, this property of a composition may be evaluated by examining the ability of the compound to deplete CD19⁺ B cells in vitro by assays known to the skilled practitioner. A therapeutically effective amount of a therapeutic compound may lower the clinical score, or otherwise ameliorate symptoms in a subject. One of ordinary skill in the art would be able to determine such amounts based on such factors as the subject's size, the severity of the subject's symptoms, and the particular composition or route of administration selected.

### I. Administration

A composition of the invention may be administered via one or more routes of administration using one or more of a variety of methods known in the art. As appreciated by the skilled artisan, the route and/or mode of administration will vary depending upon the desired results. Preferred routes of administration for PEGylated T-BsAb of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, a PEGylated T-BsAb of the invention may be administered via a non-parenteral route, such as a topical, epidermal or mucosal route of administration, for example, intranasally, orally, vaginally, rectally, sublingually or topically.

The active compounds may be prepared with carriers that will protect the compound against rapid release, such as a controlled release formulation, including implants, transdermal patches, and microencapsulated delivery systems. Biodegradable, biocompatible polymers may be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, 1978, J. R. Robinson, ed., Marcel Dekker, Inc., New York.

Therapeutic compositions may be administered with medical devices known in the art. For example, a therapeutic composition of the invention may be administered with a needleless hypodermic injection device, such as the devices disclosed in U.S. Pat. Nos. 5,399,163, 5,383,851, 5,312,335, 5,064,413, 4,941,880, 4,790,824, and 4,596,556. Examples of well-known implants and modules useful in the present invention include those described in U.S. Pat. Nos. 4,487,603, 4,486,194, 4,447,233, 4,447,224, 4,439,196, and 4,475,196. These patents are incorporated herein by reference. Many other such implants, delivery systems, and modules are known to those skilled in the art.

### J. Treatment

The diseases to be treated with PEGylated T-BsAbs can be any autoimmune diseases including, but not limit to pemphigus, neuromyelitis optica/neuromyelitis optica-spectrum disorders (NOD/NMOD), multiple sclerosis (MS), ANCA associated vasculitis, rheumatoid arthritis (RA), Crohn's disease, inflammatory bowel disease (IBD) and systemic lupus erythematosus (SLE), asthma, psoriasis, atopic dermatitis, erythematosus, type 1 diabetes.

In one aspect of the invention, the autoimmune disease is multiple sclerosis (MS).

In another aspect, the autoimmune diseases are refractory or resistant to any previous therapies as list below. For purposes of this invention, refractory or resistant autoimmune diseases defined as those do not respond to previous therapies or treatments. The autoimmune diseases can be resistant or refractory at the beginning of treatment, or they may become resistant or refractory during treatment. Refractory autoimmune diseases include those do not respond at the onset of treatment or respond initially for a short period but failed to respond afterword. Refractory autoimmune diseases also include those respond to treatment with one of conventional therapies but fail to respond to subsequent rounds of therapies. For purposes of the invention, refractory autoimmune diseases also encompass those appear to be effective by the treatment, but relapse within up to one year, sometimes within up to five years or longer after treatment is discontinued. The conventional therapies can employ small molecule agents such as Tecfidera, Gilenya, Tysabri, Aubagio, Mavenclad; peptide agent such as Copaxone, recombinant polypeptides or proteins such as IFN-β-1a, IFN-β-1b, anti-CD52 antibodies (Alemtuzumab, Alemtuzumab), Natalizumab; B cell depletion agents such as anti-CD19 agents (Inebilizumab, Obexelimab in Phase II), anti-CD20 agents (Rituximab, ocrelizumab, ofatumumab) or combination thereof. For easy of description and not limitation, it will be understood that the refractory autoimmune diseases are interchangeable with resistant autoimmune diseases.

For purposes of the present invention, successful treatment of resistant or refractory autoimmune diseases shall be understood to mean that resistant or refractory symptoms or conditions are prevented, minimized or attenuated during and/or after treatment, when compared to that observed in the absence of the treatment described herein. The minimized, attenuated or prevented refractory conditions can be confined for example by clinical scores contemplated by the artisans in the field. In one example, successful treatment of refractory or resistant MS autoimmune diseases shall be deemed to occur when the score contemplated by the artisans in the field is below the one observed in the absence of the treatment described herein. In some aspects, the resistant or refractory autoimmune diseases can be one or more of: pemphigus, neuromyelitis optica/neuromyelitis optica-spectrum disorders (NOD/NMOD), and multiple sclerosis (MS), to systemic diseases such as ANCA associated vasculitis, rheumatoid arthritis (RA), Crohn's disease, Inflammatory bowel disease (IBD) and systemic lupus erythematosus (SLE).

In one particular aspect, the resistant or refractory autoimmune disease is multiple sclerosis (MS).

The present invention provides methods of treating autoimmune diseases which are resistant or refractory to small molecule, peptide agents, protein biologics, B cell depletion agents. In one preferred aspect, the present invention provides methods of treating autoimmune diseases which are resistant or refractory to B cell depletion agents. In more preferred aspect, the present invention provides methods of treating MS which are resistant or refractory to above mentioned conventional therapies. In still more preferred aspect, the present invention provides methods of treating MS which are resistant or refractory to B cell depletion therapies.

In alternative aspects of the invention, the method of treatment of the present invention includes administering an effective amount of the compounds described herein to a mammal with autoimmune diseases. In another aspect, the method of treatment of the present invention includes administering an effective amount of the compounds described herein to a mammal with resistance or refractory autoimmune diseases. In yet another aspect, the present invention provides methods of treating autoimmune diseases which are resistant or refractory to small molecule, peptide agents, protein biologics, B cell depletion agents in combination with a second agent.

In still another aspect, the treatment of the present invention includes administering an effective amount of the compounds described herein alone or in combination, simultaneously or sequentially, with a second small molecule, peptide agent, protein biologic, and/or B cell depletion agent. The PEGylated T-BsAbs can be administered concurrently with another agent or after the administration of another agent. Thus, the compounds employed in the present invention can be administered during or after treatment of the second agent. For example, a non-liming list of the second agents includes: small molecular agents such as Tecfidera, Gilenya, Tysabri, Aubagio, Mavenclad; peptide agent such as Copaxone, protein biologics such as IFN-β-1a, IFN-β-1b, anti-CD52 antibodies (Alemtuzumab, Alemtuzumab), Natalizumab; B cell depletion agents such as anti-CD19 agents (Inebilizumab, Obexelimab in Phase II), anti-CD20 agents (Rituximab, ocrelizumab, ofatumumab).

In certain preferred embodiment of the present invention, the method for treating an autoimmune disease comprises administrating a compound having the following formula:

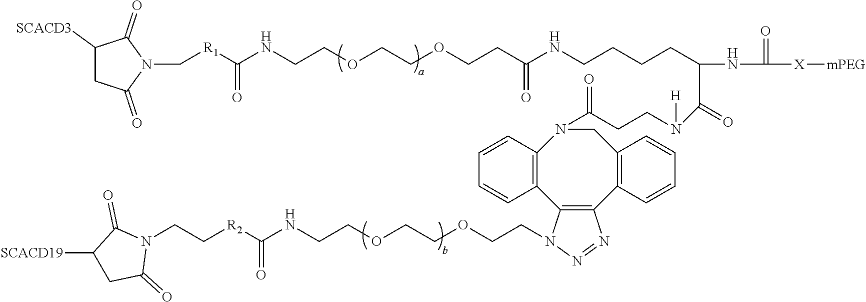

wherein the molecular weight of the mPEG is selected from 10000 to 40000; each of a and b is an integer independently selected from 1 to 20; X is selected from C, N, O; each of R₁ and R₂ is independently selected from C₁₋₁₀ alkyl or cycloalkyl.

In one particular embodiment of the present invention, the method for treating an autoimmune disease comprises administrating a compound having the following structure:

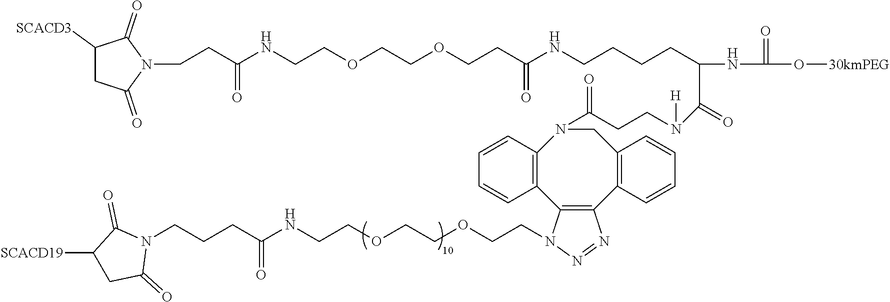

In which, the SCACD19 (anti-CD19 scFv) has following amino acid sequence:

(SEQ ID NO: 1)
DIQLTQSPASLAVSLGQRATISCKASQSVDYDGDSYLNWYQQIPGQPPK
LLIYDASNLVSGIPPRESGSGSGTDFTLNIHPVEKVDAATYHCQQSTED
PWTFGGGTKLEIKGCGGGSGGGGSGGGGSQVQLQQSGAELVRPGSSVKI
SCKASGYAFSSYWMNWVKQRPGQGLEWIGQIWPGDGDTNYNGKFKGKAT
LTADESSSTAYMQLSSLASEDSAVYFCARRETTTVGRYYYAMDYWGQGT
TVTVSSHHHHHH;

and the SCACD3 (anti-CD3 scFv) has the following amino acid sequence:

(SEQ ID NO: 2)
DIKLQQSGAELARPGASVKMSCKTSGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSVEGCGSGGSGGSGGSGGVDDIQLTQSPA
IMSASPGEKVTMTCRASSSVSYMNWYQQKSGTSPKRWIYDTSKVASGVP
YRFSGSGSGTSYSLTISSMEAEDAATYYCQQWSSNPLTFGAGTKLELKH
HHHHH

The compound having the above structure is referred to as “JY108” herein.

### K. Definition of Terms

The term “alkyl” as used herein refers to a hydrocarbon chain, typically ranging from about 1 to 25 atoms in length. Such hydrocarbon chains are preferably but not necessarily saturated and may be branched or straight chain, although typically straight chain is preferred. The term C₁₋₁₀ alkyl includes alkyl groups with 1, 2, 3, 4, 5, 6, 7, 8, 9 and 10 carbons. Similarly C₁₋₂₅ alkyl includes all alkyls with 1 to 25 carbons. Exemplary alkyl groups include methyl, ethyl, isopropyl, n-butyl, n-pentyl, 2-methyl-1-butyl, 3-pentyl, 3-methyl-3-pentyl, and the like. As used herein, “alkyl” includes cycloalkyl when three or more carbon atoms are referenced. Unless otherwise noted, an alkyl may be substituted or un-substituted.

The term “function group” or “functional group” as used herein refers to a group that may be used, under normal conditions of organic synthesis, to form a covalent linkage between the entity to which it is attached and another entity, which typically bears a further functional group. A “bifunctional linker” refers to a linker with two functional groups forms two linkages via with other moieties of a conjugate.

The term “derivative” as used herein refers to a chemically modified compound with an additional structural moiety for the purpose of introducing new functional group or tuning the properties of the original compound.

The term “protecting group” as used herein refers to a moiety that prevents or blocks reaction of a particular chemically reactive functional group in a molecule under certain reaction conditions.

The term “PEG” or “poly(ethylene glycol)” as used herein refers to poly(ethylene oxide). PEGs for use in the present invention typically comprise a structure of (CH₂CH₂O)ₙ. PEGs may have a variety of molecular weights, structures or geometries.

A PEG group may comprise a capping group that does not readily undergo chemical transformation under typical synthetic reaction conditions. Examples of capping groups include —OC₁₋₂₅ alkyl or —OAryl.

The term “linker” or “linkage” as used herein refers to an atom or a collection of atoms used to link interconnecting moieties, such as an antibody and a polymer moiety. A linker may be cleavable or noncleavable. Cleavable linkers incorporate groups or moieties that may be cleaved under certain biological or chemical conditions. Examples include enzymatically cleavable disulfide linkers, 1,4- or 1,6-benzyl elimination, trimethyl lock system, bicine-based self-cleavable system, acid-labile silyl ether linkers and other photolabile linkers.

The term “linking group” or “linkage group” as used herein refers to a functional group or moiety connecting different moieties of a compound or conjugate. Examples of a linking group include, but are not limited to, amide, ester, carbamate, ether, thioether, disulfide, hydrazone, oxime, and semicarbazide, carbodiimide, acid labile group, photolabile group, peptidase labile group and esterase labile group. For example, a linker moiety and a polymer moiety may be connected to each other via an amide or carbamate linkage group.

The terms “peptide,” “polypeptide,” and “protein” are used herein interchangeably to describe the arrangement of amino acid residues in a polymer. A peptide, polypeptide, or protein may be composed of the standard 20 naturally occurring amino acid, in addition to rare amino acids and synthetic amino acid analogs. They may be any chain of amino acids, regardless of length or post-translational modification (for example, glycosylation or phosphorylation).

A “recombinant” peptide, polypeptide, or protein refers to a peptide, polypeptide, or protein produced by recombinant DNA techniques; i.e., produced from cells transformed by an exogenous DNA construct encoding the desired peptide. A “synthetic” peptide, polypeptide, or protein refers to a peptide, polypeptide, or protein prepared by chemical synthesis. The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Within the scope of this invention are fusion proteins containing one or more of the afore-mentioned sequences and a heterologous sequence. A heterologous polypeptide, nucleic acid, or gene is one that originates from a foreign species, or, if from the same species, is substantially modified from its original form. Two fused domains or sequences are heterologous to each other if they are not adjacent to each other in a naturally occurring protein or nucleic acid.

An “isolated” peptide, polypeptide, or protein refers to a peptide, polypeptide, or protein that has been separated from other proteins, lipids, and nucleic acids with which it is naturally associated. The polypeptide/protein may constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity may be measured by any appropriate standard method, for example, by column chromatography, polyacrylamide gel electrophoresis, or HPLC analysis. An isolated polypeptide/protein described in the invention may be purified from a natural source, produced by recombinant DNA techniques, or by chemical methods.

An “antigen” refers to a substance that elicits an immunological reaction or binds to the products of that reaction. The term “epitope” refers to the region of an antigen to which an antibody or T cell binds.

As used herein, “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

As used herein, “antibody fragments”, may comprise a portion of an intact antibody, generally including the antigen binding and/or variable region of the intact antibody and/or the Fc region of an antibody which retains FcR binding capability. Examples of antibody fragments include linear antibodies; single-chain antibody molecules; and multispecific antibodies formed from antibody fragments. Preferably, the antibody fragments retain the entire constant region of an IgG heavy chain, and include an IgG light chain.

As used herein, the term “Fe fragment” or “Fe region” or “FC” is used to define a C-terminal region of an immunoglobulin heavy chain.

The term “traditional antibody” is used herein to refer to whole length monoclonal antibody or its modified version.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature, 256, p 495-497, which is incorporated herein by reference, or may be made by recombinant DNA methods (see, e.g., U.S. Pat. No. 4,816,567, which is incorporated herein by reference).

The monoclonal antibodies may also be isolated from phage antibody libraries using the techniques described in Clackson et al., 1991, Nature, 352, p 624-628 and Marks et al., 1991, J Mol Biol, 222, p 581-597, for example, each of which is incorporated herein by reference.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; Morrison et al., 1984, Proc Natl Acad Sci USA, 81, p 6851-6855; Neuberger et al., 1984, Nature, 312, p 604-608; Takeda et al., 1985, Nature, 314, p 452-454; International Patent Application No. PCT/GB85/00392, each of which is incorporated herein by reference).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody optionally also will comprise at least a portion of an immunoglobulin constant region (Fc), typically that of a human immunoglobulin. For further details, see Jones et al., 1986, Nature, 321, p 522-525 (1986); Riechmann et al., 1988, Nature, 332, p 323-327; Presta, 2003, Curr Op Struct Biol, 13(4), p 519-525; U.S. Pat. No. 5,225,539, each of which is incorporated herein by reference. “Human antibodies” refer to any antibody with fully human sequences, such as might be obtained from a human hybridoma, human phage display library or transgenic mouse expressing human antibody sequences.

The term “pharmaceutical composition” refers to the combination of an active agent with a carrier, inert or active, making the composition especially suitable for diagnostic or therapeutic use in vivo or ex vivo.

As used herein, “pharmaceutically acceptable carrier” includes any and all solvents, dispersion media, coatings, antibacterial and antifungal agents, isotonic and absorption delaying agents, and the like that are physiologically compatible. A “pharmaceutically acceptable carrier,” after administered to or upon a subject, does not cause undesirable physiological effects. The carrier in the pharmaceutical composition should be “acceptable” also in the sense that it is compatible with the active ingredient and may be capable of stabilizing it. One or more solubilizing agents may be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate.

Additional suitable pharmaceutical carriers and diluents, as well as pharmaceutical necessities for their use, are described in Remington's Pharmaceutical Sciences. Preferably, the carrier is suitable for intravenous, intramuscular, subcutaneous, parenteral, spinal or epidermal administration (e.g., by injection or infusion). The therapeutic compounds may include one or more pharmaceutically acceptable salts. A “pharmaceutically acceptable salt” refers to a salt that retains the desired biological activity of the parent compound and does not impart any undesired toxicological effects (see e.g., Berge, S. M., et al. 1977, J. Pharm. Sci. 66, p 1-19).

As used herein, “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder.

An “effective amount” refers to the amount of an active compound/agent that is required to confer a therapeutic effect on a treated subject. Effective doses will vary, as recognized by those skilled in the art, depending on the types of conditions treated, route of administration, excipient usage, and the possibility of co-usage with other therapeutic treatment. A therapeutically effective amount of a combination to treat an autoimmune disease is an amount that will cause, for example, a reduction of clinical score or slow the progress of the symptoms, as compared to untreated subjects.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” generally refers to plus or minus 10% of the indicated number. For example, “about 10%” may indicate a range of 9% to 11%, and “about 1” may mean from 0.9-1.1. Other meanings of “about” may be apparent from the context, such as rounding off, in such cases, for example “about 1” may also mean from 0.5 to 1.4.

### EXAMPLES

The following examples serve to provide further appreciation of the invention but are not meant in any way to restrict the effective scope of the invention.

### Example 1: Target Binding of PEGylated BsAb Anti-CD3× Anti-CD19 (JY108)

Since T-BsAb (which targets CD3 and a tumor associated antigen) with strong affinity to TCR, such as blinatumomab, is associated with excessive cytokine release which often results in severe CRS in clinical, target binding of JY108 was evaluated by BiaCore assay and ELISA assay.

### 1) BiaCore Assay

Equipment: BiaCore

Samples: anti-CD3 scFv (SCACD3), anti-CD3 scFv-PEG (anti-CD3-PEG), anti-CD19 scFv (SCACD19), anti-CD19 scFv-Linker (anti-CD19-Linker), JY108 and Blinatumomab. The anti-CD3 scFv (SCACD3) has the amino acid sequence set forth in SEQ ID NO: 2. The anti-CD19 scFv (SCACD19) has the amino acid sequence set forth in SEQ ID NO: 1. Methods for preparation of theses samples were described in detail in WO 2018/075308 A1.

General procedure: Diluted human CD19 (AcroBiosystems, Cat. CD19-H5259) or human CD3E&3D (AcroBiosystems, Cat. CDD-H52W0) were captured on the sensor chip through Fc capture method. The capture time for human CD3E & CD3D and for human CD19 was set for 12 s and 12-30 s respectively and capture flow rate was 10 L/min. Each sample tested was serially diluted into 6-8 concentrations. The association time and dissociation time for the analytes were set for 180 s and 420 s respectively, and the flow rate for the analyte was set at 30 L/min. For surface regeneration, 10 mM Glycine-HCl buffer was used; the contact time was set for 30 s and the flow rate was 30 L/min. Flow cell 1 and blank injection of buffer in each cycle were used as double references for Response Units subtraction. All data were processed using BiaCore T2000 Evaluation software (version 3.1). The binding kinetic data are shown in Table 1.

As showed in Table 1, KD (binding affinity) for JY108 to human CD3E&CD3D is 3.31×10⁻⁸M, while KD for blinatumomab is 1.14×10⁻⁹M. The affinity of JY108 is around 30 times weaker than that of blinatumomab. The reduced affinity of JY108 to CD3 is probably caused by the hindrance effect of large PEG since the affinity of anti-CD3 scFv-PEG to the target is also weaker (14 times) than that of non-PEGylated anti-CD3 scFv.

Similarly, KD for JY108 to human CD19 is 1.44×10⁻⁹M while KD for blinatumomab is 2.07×10⁻¹⁰M. Again, hindrance effect caused by PEG probably have contributed to the weakened affinity of JY108 to human CD19. As expected, KD for anti-CD19 scFv-linker (linker is a much smaller PEG₁₁) to human CD19 is 1.11×10⁻¹⁰M, which is similar to KD for anti-CD19 scFv (6.53×10⁻¹¹M).

TABLE 1
Affinity measurement of antibodies to human CD19/human CD3E&3D

| | | ka | kd | KD | Rmax | Chi² |
|---|---|---|---|---|---|---|
| Ligand | Analyte | (1/Ms) | (1/s) | (M) | (RU) | (RU²) |
| human CD3E&3D | anti-CD3 scFv | 2.10E+05 | 2.43E−03 | 1.16E−08 | 50.49 | 1.91 |
| human CD3E&3D | anti-CD3 scFv-PEG | 6.25E+04 | 1.02E−02 | 1.63E−07 | 55.49 | 0.0859 |
| human CD3E&3D | JY108 | 2.67E+05 | 8.84E−03 | 3.31E−08 | 51.86 | 1.29 |
| human CD3E&3D | Blinatumomab | 1.08E+06 | 1.23E−03 | 1.14E−09 | 53.32 | 2.51 |
| human CD19 | anti-CD19 scFv | 5.26E+06 | 3.43E−04 | 6.53E−11 | 9.14 | 0.0068 |
| human CD19 | anti-CD19 scFv-Linker | 2.79E+06 | 3.09E−04 | 1.11E−10 | 7.50 | 0.00332 |
| human CD19 | JY108 | 3.92E+05 | 5.66E−04 | 1.44E−09 | 12.67 | 0.0164 |
| human CD19 | Blinatumomab | 1.92E+06 | 3.97E−04 | 2.07E−10 | 11.83 | 0.00892 |

### 2) ELISA Assay

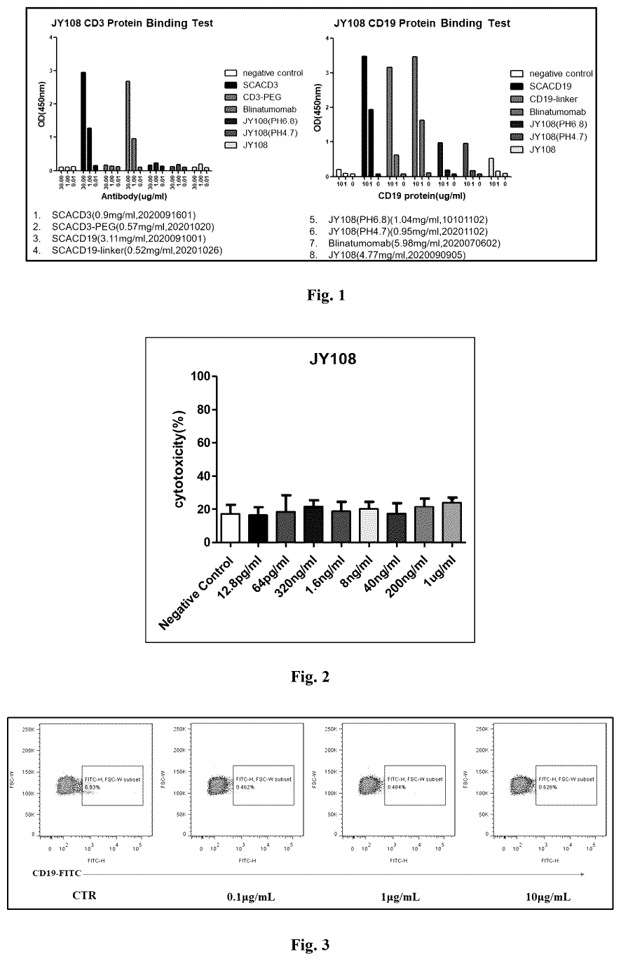

To evaluate binding to human CD3E&CD3D protein, the sample solutions of SCACD3, CD3-PEG (PEGylated SCACD3), blinatumomab and 3 lots of JY108 (2 lots in pH6.8 and 1 in pH4.7 buffer respectively) were prepared with CBS with four concentrations of 30, 1 and 0.01 μg/mL for each sample. Plates were coated with SCACD3, CD3-PEG and JY108 respectively at 100 μl/well. Similarly, to evaluate binding to human CD19 protein, the sample solutions of SCACD19, CD19-linker, blinatumomab and three lots of JY108 (2 lots in pH6.8 and 1 in pH4.7 buffer respectively) were prepared with CBS with two concentrations of 10, 1 μg/mL for each sample and coated to plates with 100 μl/well. After coating, each plate was washed for 3 times with PBST (200 μL/well). Next, each plate was blocked with 5% BSA in PBST at 37° C. for 2 hours. After blocking, each plate was washed with PBST for 3 times followed by the addition of either 1 g, 0.1 μg, and 0 μg human CD19-Fc protein (Acrobiosystems, Code: CD9-H5259) in 0.5% BSA in PBST (10 μg/mL, 1 μg/mL, 0 μg/mL, 100 μL/well) or 1 g human CD3E&CD3D (Acrobiosytems, Code: CDD-H52W0) in 0.5% BSA in PBST (10 μg/mL, 100 μl/well) to corresponding wells. The plates were incubated at 37° C. for 1 hour followed by washing with PBST for 3 times. 100 μL of 1 g/mL HRP conjugated anti-human IgG (GenScript, Code: A01854) was then added to each well. The plates were incubated at 37° C. for 1.5 hours. After incubation, each plate was washed with PBST for 3 times followed by the addition of 100 μL of TMB to each well. The development reaction for each plate was set in dark for 15 minutes and terminated with 100 μL/per well of stop solution. Each plate was read at OD450 nm with a microplate reader and the results are shown in FIG. 1.

The results in FIG. 1 demonstrated that, at the coating concentration of 1 μg/ml, the affinity of tested samples to human CD3E&CD3D could be ranked as: SCACD3>blinatumomab>JY108>SCACD3-PEG. Similar ranking for samples binding to human CD19 was also observed (SCACD19>blinatumomab>JY108>SCACD19-linker).

Conclusion: the results from Biacore assay and ELISA assay are consistent and demonstrated that PEGylated T-BsAb JY108 has significantly reduced affinity to both CD3 and CD19. The decreased affinities through PEGylation observed in these two assays are expected to bring beneficial effects for JY108, e.g. with less cytokine release.

### Example 2: In Vitro Efficacy of JY108 to CD19⁺ Cell Lines and Normal B Cells

To test the efficacy of PEGylated T-BsAb JY108 towards a variety of target cells despite reduced binding to the targets, following experiments were performed.

General procedure: expanded effector cells at 4×10⁴, 10×10⁴ or 20×10⁴ (E:T ratios would be 2:1, 5:1 or 10:1 respectively) in 100 μl medium were incubated with indicated doses of JY108 for half an hour at room temperature. 2×10⁴ CD19⁺ or CD19⁻ target cells (Raji, Nalm-6, REH or K562) were added and incubated at 37° C. for 24 hours or as indicated. For effector-cell-only wells, 100 μL medium without target cells were added and for target-cells-only wells, 100 μL medium without effector cells were added. Each well was added with 20 μL of MTS reagent and incubated at 37° C. by following the manufacturer's manual. After incubation, absorbance at OD₄₉₀ nm was recorded, and the percentage of dead cells was calculated based on the formula:

Cytotoxicity %=1−(OD_{Experimental}−OD_{PBMC})/(OD_{Target}−OD_{medium})
wherein ODₑₓₚₑᵣᵢₘₑₙₜₐₗ refers to the OD₄₉₀ of the wells containing JY108, effector cells and targets at designed E:T ratio. OD_{PBMC} refers to the OD₄₉₀ of effector-cell-only with indicated JY108 doses with no target cells. OD_{target} refers to the OD₄₉₀ of target-cells-only without JY108 and effector cells. OD medium refers to the OD₄₉₀ of the equal volume of medium with no JY108, effector cells or target cells. This procedure was used for in vitro efficacy assays of JY108 to all cell lines, except to B cells in PBMC, in which case fluorescent antibody staining followed by flowcytometry detection was used.

### 1) No Drug-Specific Cytotoxicity to CD19⁻ Cells by JY108

To test whether JY108 targets CD19⁻ cells, a leukemia cell line K562 with negative CD19 expression was used in this experiment. The results in FIG. 2 showed that even at a concentration of 1 μg/mL with E:T=5:1, there was no significant difference between the cell killing effects in the presence and absence of JY108 (negative control has no JY108). The results indicate that JY108 is safe and does not cause drug-specific cytotoxicity to CD19 negative cells.

2) Elimination of B Cells in PBMC from Healthy Donors by JY108

To test whether JY108 can eliminate CD19-positive cells from PBMC, JY108 in 4 different concentrations (0 μg/ml in control, 0.1 μg/ml, 1 μg/ml, and 10 μg/ml) was added to PBMC and incubated at 37° C. for 24 hours. After incubation, PBMC samples were stained with 1 μl FITC labeled anti-CD19 for 30 minutes followed by detection with Flowcytometry. The results were shown in FIG. 3. Comparing to the control sample (without JY108) of 6.93% of CD19 positive cells, JY108 significantly decreased CD19 positive cells to the background level at the concentration of 0.1 μg/ml. This result clearly showed that JY108 can efficiently eliminate CD19 positive cells from PBMC.

### 3) E:T Ratio Effect on JY108 Efficacies

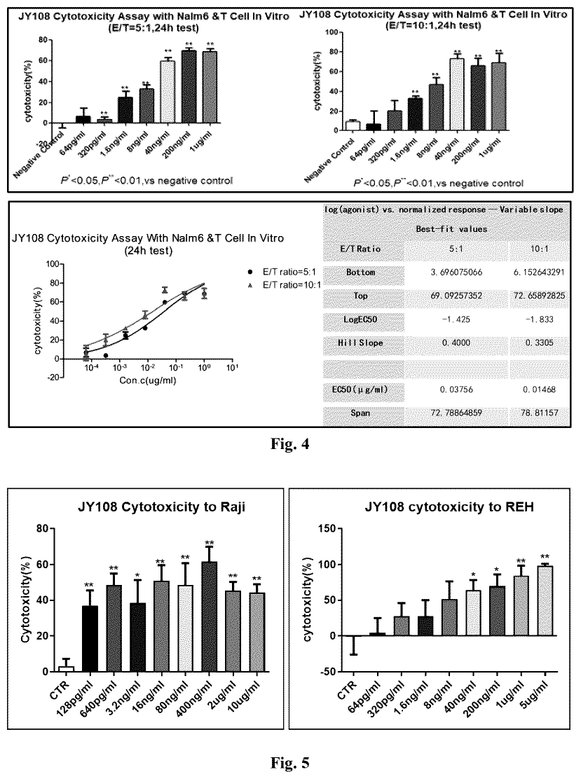

With different E:T ratios, JY108 showed different toxicity against the same target cells. The results in FIG. 4 showed that EC₅₀ of JY108 against Nalm-6 killing at E:T=5:1 was 37.56 ng/mL, while EC₅₀ of JY108 at E:T=10:1 was 14.68 ng/ml. The result showed that with more effector cells, less JY108 was required to achieve desired cell killing. However, the increased cytotoxicity of JY108 with increased E:T ratio plateaued when other parameters kept unchanged (data not shown).

### 4) Cell Lines Effect on JY108 Efficacies

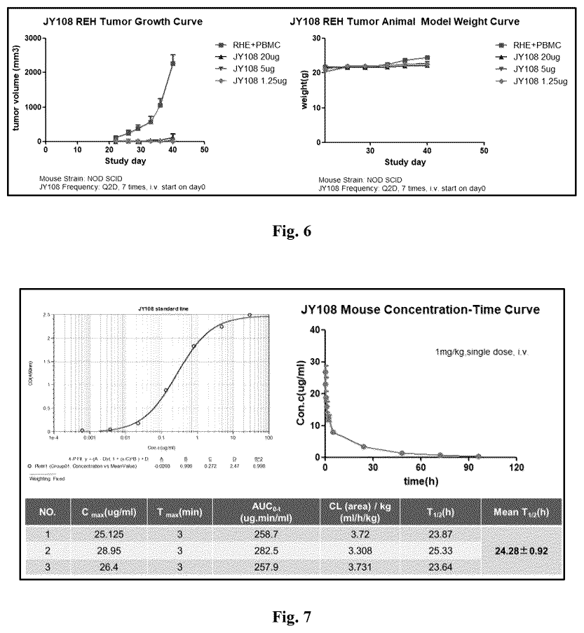

JY108 has different killing effects on different CD19⁺ cell lines (FIG. 5). EC₅₀ of JY108 against REH was 10.38 ng/mL while EC₅₀ of JY108 against Raji cells was 159.3 ng/mL at the experimental settings.

Conclusion: the EC₅₀ of JY108 was found to be at the ng/mL level in all assays, ranging from single digit ng/mL to hundreds of ng/mL, with different E:T ratios, different PBMC donors, different durations of action, and different CD19-positive cell lines. The In vitro killing of pathologic CD19⁺ cells is drug specific and consistent with the expectations of JY108 drug design.

### Example 3: In Vivo Efficacy of JY108 Against Pre-B ALL Tumor Model

To test the in vivo efficacy of JY108, the REH tumor cells were collected and counted followed by the adjustment of cell concentration to 10×10⁷/mL. The expanded PBMC cells were adjusted to 30×10⁷/mL. Equal volume of expanded BMC cells and REH tumor cells were mixed right before being inoculated to the animals. JY108 was prepared with sterilized PBS to the concentration of 200 μg/mL, 50 μg/ml, and 12.5 μg/ml respectively. The mice were randomly divided with 5 mice/group. Tail vein administration (100 μL/mice) of the prepared JY108 was performed. After injection of JY108, the REH and expanded PBMC cells mixed in equal volume were subcutaneously inoculated into the mice (200 μL/mice).

The results in FIG. 6 showed that JY108 at the dose of 1.25 ag could effectively inhibit REH tumor growth. There was no significant difference of body weight between the mice in experimental groups and those in the control groups, suggesting that JY108 at the experimental doses did not cause toxic effects on the animals.

### Example 4: Pharmacokinetic Property of JY108

JY108 comprises His tagged SCACD3 and SCACD19 without his tag. Therefore, anti-His tag antibody and CD19 antigen can be used in ELISA to detect intact JY108 molecule. In the similar ELISA assay described in example 1, human CD19-Fc is used as the coating agent at 1 μg/well and anti-His is used as the detection antibody. The results shown in FIG. 7 demonstrated a T_{1/2} of 24.28 hr. for JY108 in wild type C57BL/6.

The half-life of JY108 in the tested animals is much longer than the control drug blinatumomab which is about 2 h as reported. Since the metabolism of PEGylated protein in rodents is about 5 times faster than that in human (US 2011/0112021 A1), the half-life of JY108 in the human body is therefore expected to be greater than 120 hours, which will greatly benefit in clinical efficacy of JY108.

### Example 5: Selection of Humanized Transgenic Mice (hCD3ehCD19) as the Animal Models for Toxicity Evaluation of JY108

In this study, a humanized transgenic mice for both target genes (CD38 and CD19) was constructed and used to evaluate toxicity and in vivo efficacy of the molecule.

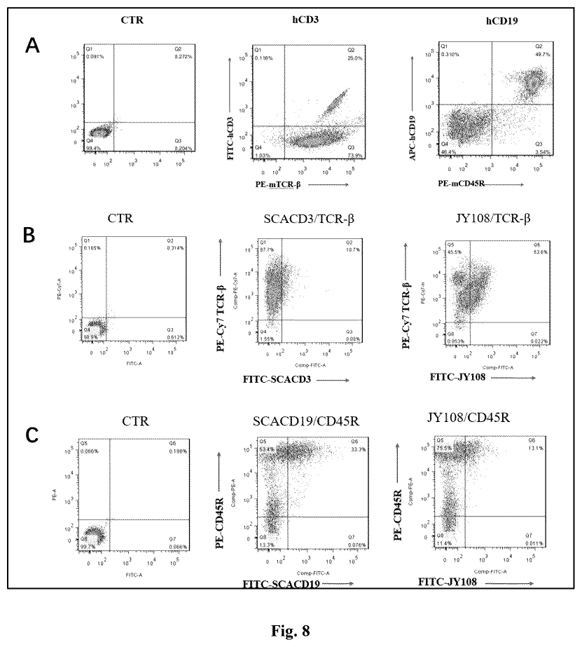

### 1) Binding of JY108 to Targets of Animal Model (FIG. 8)

The SCACD3, SCACD19 and JY108 were FITC labeled respectively according to the vendors' protocol of Lightning-Link®. The spleen tissues of hCD3ehCD19 transgenic mice were collected and grinded in lymphocyte isolate solution followed by centrifuging to obtain suspended mice lymphocytes. After the lymphocytes were collected and washed, cold FACS buffer (PBS with 3% FBS) was used to adjust the cell suspension to 5×10⁶ cells/ml. The resuspended cells were aliquoted in 100 μL/tube. The FICT (or PE, or APC) labeled antibodies were added to the cell suspension and the reaction mixtures were incubated in dark at 4° C. for 30 minutes. Subsequently, the cells were washed for 3 times with FACS buffer by centrifuging at 400 g for 5 min on each wash. Before using flow cytometry to analyze the cells, 500 μL FACS buffer (cold PBS supplemented with 3% FBS) were used to resuspend cells.

In FIG. 8A, the flow cytometry results showed that recombinant human CD3 (FITC-hCD3) is expressed on murine T cells (PE-mTCR-b), and recombinant human CD19 (APC-hCD19) is expressed on murine B cells (PE-mCD45). In FIG. 8B, the results demonstrated that 10.7% spleen cells from the transgenic mice were double stained by both murine TCR-b and FITC labelled SCACD3, and 53.6% spleen cells from the transgenic mice were double positive for both murine TCR-b and FITC labelled JY108. In FIG. 8C, 33.3% spleen cells from the transgenic mice were double positive for both murine CD45R and SCACD19, and 13.1% spleen cells from the transgenic mice were double positive for both murine CD45R and JY108. The combined results from FIGS. 8A, 8B and 8C demonstrated that the lymphocytes from the hCD3ehCD19 transgenic mice were specifically bound by SCACD3, SCACD19, and JY108.

2) Spleen Cells from Transgenic Mice (hCD3hCD19) as Effectors for JY108

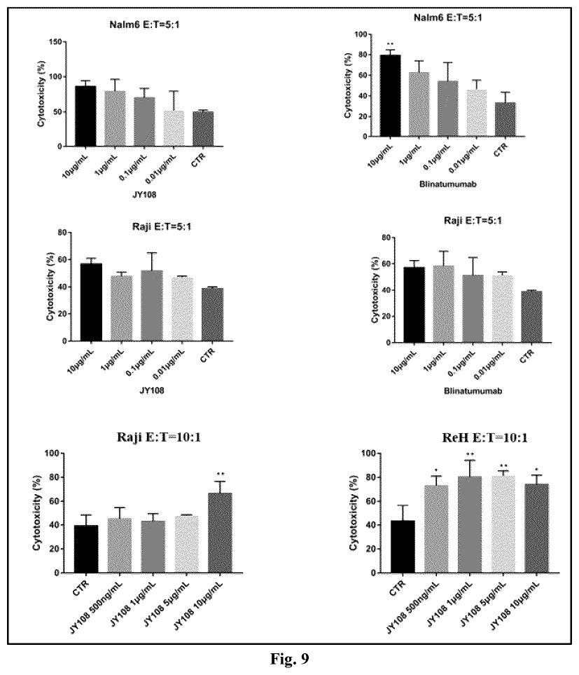

T cells from the transgenic mice have been evaluated and it was found that these can be used as effector cells as expected in in vitro cytotoxicity assay of JY108. Similar to the results in example 2, the spleen cells from hCD3ehCD19 transgenic mice could also effectively kill Raji, Nalm-6, and ReH target cells, as shown in FIG. 9.

Conclusion: the above results demonstrated that the transgenic mice hCD3hCD19 are animal models that could be used for toxicity evaluation of JY108.

### Example 6: No Acute Toxicity of JY108 at Single Dose of 10 mg/kg

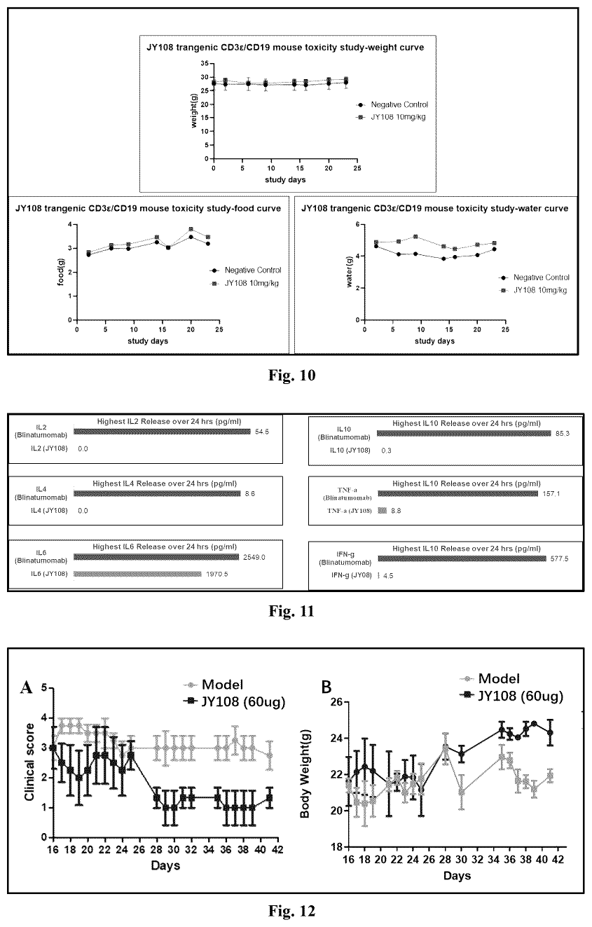

The hCD3ehCD19 transgenic mice were dosed at 10 mg/kg of JY-108 intravenously (n=3, single dose) for an acute toxicity observation. Compared with the negative control group, hCD3ehCD19 homozygous transgenic mice of the experiment group have no significant changes in body weight (p>0.05), food intake, but a slight increase in water consumption (FIG. 10).

### Example 7: Less Cytokine Release from Human Whole Blood by JY108

Cytokine storm or cytokine release syndrome is one of the major clinical side effects of T cell engaged antibody drugs (e.g., blinatumomab). To assess whether reduced affinities of JY108 could lead to less cytokine release and improved safety profile as compared to the control drug blinatumomab, both JY108 and blinatumomab at different concentrations (0.0005 μg/ml, 0.005 μg/ml, 0.05 μg/ml, 0.5 μg/ml, and 5 μg/ml) were incubated with the human whole blood (HWB) from 2 healthy donors for 24 hours. The released cytokines at 2 hours, 6 hours and 24 hours of incubation were measured using BD™ Cytometric Bead Array (CBA) Human Th1/Th2 (Catalog No:551809) by flow microsphere technique. The average value of the two donors were calculated. The highest value for each cytokine over 24 hrs were shown in FIG. 11. The results showed that cytokine release including IL2, IL6, TNF, IFN-γ, and IL10 induced by JY108 were all lower than those induced by the control drug blinatumomab. Therefore, it can be predicted that JY108 would cause significantly lower cytokine storms and associated side effects in vivo than blinatumomab.

### Example 8: JY108 Significantly Ameliorated EAE

The purpose of this study was to test the efficacy of JY108 on experimental autoimmune encephalomyelitis (EAE), an autoimmune disease model for MS.

### 1) Generation of EAE Model

The EAE model was generated on C57BL/6 transgenic mice (hCD3ehCD19 homozygotes) by following a standard protocol. Before generation of the model, 100 μg/mL stock solution of pertussis toxin (PTX) was prepared in PBS and kept at 4° C. On the day of each injection, PTX stock solution was diluted to 1 μg/mL working solution in PBS. On Day 0, equal volume of MOG₁₋₁₂₅ protein (2 mg/mL) and Complete Freund's Adjuvant (CFA, 4 mg/mL) were thoroughly emulsified in a homogenizer until the mixture became homogeneous. Then the emulsion of MOG₁₋₁₂₅ and CFA were subcutaneously injected into mice at both sides of thoracic and abdomen part (150 μL/mice) followed by the tail vein injection of PTX at 200 μL/mice. On Day 2, a second tail vein injection of PTX at 200 μL/mice was performed. Body weight of each mouse was measured daily and animal disease symptoms were evaluated with clinical scores (CS) according to Kono's 5-score criteria (Grade 1: Limp tail; Grade 2: Limp tail and hind limb weakness; Grade 3: Partial hind limb paralysis; Grade 4: Complete hind limb paralysis; Grade 5: Moribund state) (Hou, Y. et al. 2013, Stem Cell Res Ther 4, 77).

### 2) Significantly Reducing EAE Symptoms by JY108

EAE symptoms began to appear in the transgenic mice on Day 11 and continue worsening with time. On Day 16, the CSs reached 3. EAE model mice with symptoms were then grouped randomly on Day 16 so that the experimental and control groups were at similar clinical scores and body weights (n=4 for each group). The experimental group was tail vein administered with JY108 at 60 μg/animal every other day (7 times in total) and stopped dosing at Day 28, while the control group (model group in FIG. 12) was i.v. injected with vehicle. The disease symptoms of two groups of mice were monitored and their CSs were recorded on daily basis.

As shown in FIG. 12, the JY108 group showed significant relief of symptoms after the initial administrations and rebounded later, and then remained stable at a relatively low CS. The mice in model group (control group) without JY108 showed a worsening trend of symptoms at the beginning of the grouping, followed by a slight remission, but symptom scores remained stable at a relatively high level. The weight curves appear to correlate well with the clinical symptom profile of EAE: low body weight when the condition was severe and high body weight when the condition was in remission. It is possible that the EAE condition affects mobility of mice, including the ability to reach food.

### 3) LFB Staining of Histopathological Sections

To further support the observed evidence of ameliorating EAE symptoms, the spinal cord and brain tissues of mice were stained according to the pathological test procedures. Nikon (Eclipse Ci-L) microscope and 3DHISTECH slice scanner (PANNORAMIC DESK/MIDI/250/1000) with CaseViewer2.2 were used to acquire images.

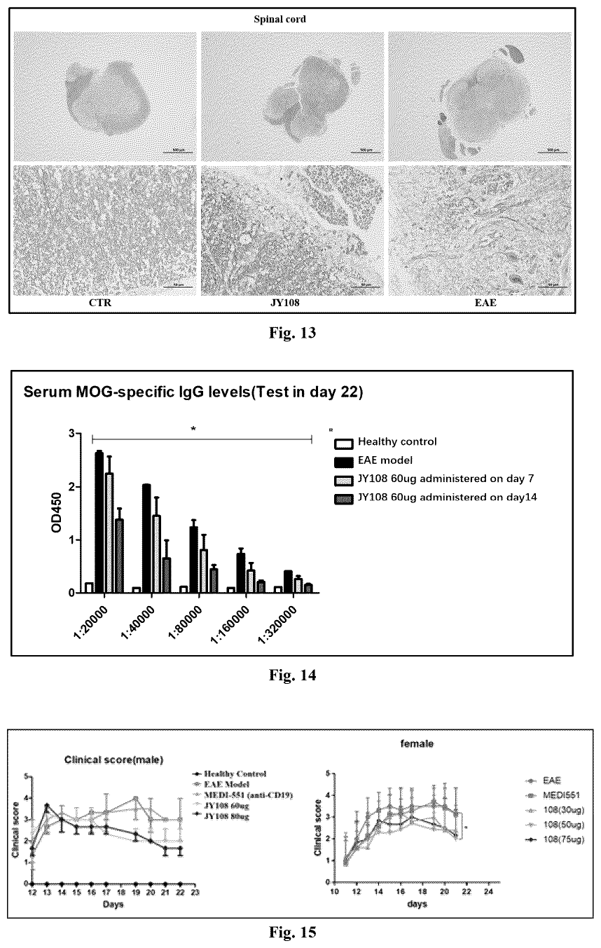

LFB (Luxol Fast Blue) dye can be attracted by myelin sheath lipoprotein to specifically stain CNS tissues. The results are shown in FIG. 13. For spinal cord, the LFB staining results showed that the myelin of white matter of the healthy animals were uniform and could be evenly stained, and no obvious demyelination was observed. In the model group, a large area around the white matter disappeared, the LFB stained myelin became lighter and discontinuous, indicating many nerve fibers were demyelinated. In contrast, the spinal cord from JY108 treated animals showed more LFB stained area and the staining were more continuous than that of the EAE model group. The results indicated that the severity of demyelination in the model is much reduced by JY108.

Based on the results of improvement of clinical symptoms, body weight changes and pathological section analysis of tissue LFB staining, the conclusion can be reached that JY108 is effective in ameliorating EAE symptoms.

### 4) MOG Specific Autoantibody Depletion by JY108

To further understand the efficacy of JY108 in EAE mice, MOG specific autoantibodies from animals on day 22 were examined. The similar procedures to ELISA method described in Examples 1 and 4 were used for this assay except the MOG₁₁₂₅ protein was used to coat plates. The results shown in FIG. 14 demonstrated that JY108 efficiently reduced MOG specific antibody levels in the serum (FIG. 14).

### Example 9: Head-to-Head Comparison of JY108 to MEDI-551

MEDI-551 (Inebilizumab) is an anti-CD19 monoclonal antibody currently under clinical trial for treating MS. The pre-clinical results showed that MEDI-551 is more effective than marketed monoclonal anti-CD20 antibody in EAE model. For head-to-head comparison, EAE model in this experiment were constructed in the same way as described in Example 8 and MEDI-551 treated group (a single i.v. injection of 250 μg/animal) was included in the experiment. On Day 12 after generation of the EAE model, the male animals were randomly grouped into 4 groups (n=7): MEDI-551 treated group, model group (control), two groups treated with JY108 at 60 μg/animal and at 80 μg/animal with 5 doses in total (Day 14, Day 15, Day 17, Day 19 and Day 21). The clinical scores were recorded for each animal on daily basis (except Day 18). Female mice were grouped similarly (n=7) after day 11 of EAE induction and treated with JY108 (30, 50, 75 μg/animal) and MEDI551 (250 μg/animal).

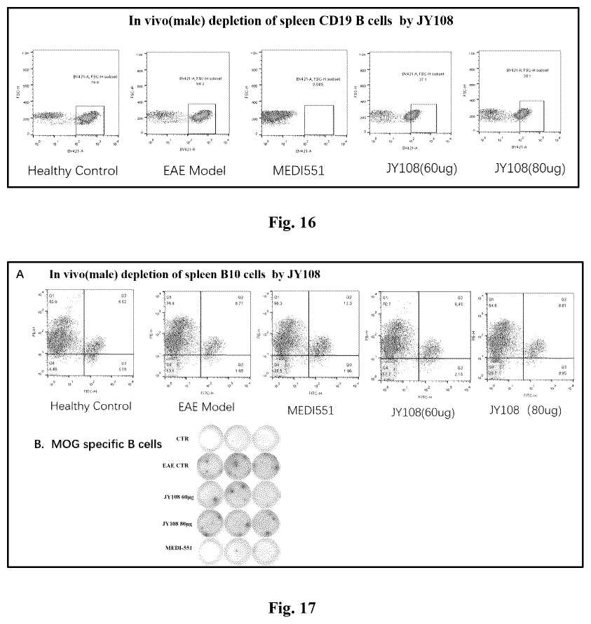

As shown in FIG. 15, JY108 dosed at both 60 μg/animal and 80 μg/animal significantly ameliorated EAE symptoms of male mice when compared with the vehicle treated EAE model group. Furthermore, JY108 at 80 μg/animal was more effective than at 60 μg/animal for ameliorating EAE symptoms of male mice. Similar results were also obtained for female EAE mice treated with 30, 50, and 75 μg of JY108/animal while treatment with MEDI551 was not effective. Although MEDI-551 administered on day 7 after EAE induction was reported to be able to delay the EAE symptoms of male mice (Chen, D. et al. 2014, J Immunol 193, 4823-4832), surprisingly, the same compound administered on day 14 in this experiment did not show any therapeutic effect for the severe sicked EAE animals (p>0.05) (FIG. 15).

### Example 10: Mechanisms of JY108 in Ameliorating EAE

### 1) Partial Removal of CD19+ B Cell by JY108

To better illustrate the mechanism of JY108 in ameliorating EAE symptoms, the spleen B cells in the EAE experiment were examined on day 15, using the same method described in Example 9. The results are shown in FIG. 16. Surprisingly, while MEDI-551 almost completely eliminated CD19⁺ B cells, JY108 only partially removed the CD19⁺ B cells: from 68.2% in the EAE models to 37.1% in JY108 treated group at 60 μg/animal, and 39.1% at 80 μg/animal.

### 2) B_{reg} Cells are Resistant to Both MEDI-551 and JY108

JY108 demonstrated higher efficiency in ameliorating EAE symptoms than MEDI-551 (FIG. 15), but it was not as effective as MEDI-551 in depleting total CD19⁺ B cells (FIG. 16). The seemed contradictory results prompted us to see what happened to other related immune cells after JY108 treatment. Using the method described in Example 5, the spleen cells derived from the animals in the EAE experiment were double stained with PE-anti-CD19 and FITC-B10 (a marker for Breg) and counted via flowcytometry. As showed in FIG. 17A, the levels of immune inhibitory Breg cells were similar among all the five groups, which indicated that both JY108 and MEDI551 had little effect on Breg cells at the time when samples were collected.

Since MOG specific B cells are the major source of autoantibodies for CNS myelination damage, ELISPOT was performed by following the protocol provided by the vendor's manual to compare the residual MOG specific B cells in the spinal cord of the tested animals. The results shown FIG. 17B were consistent with those in FIG. 16, indicating that both JY108 and MEDI551 depleted MOG specific B cells effectively.

Conclusion: the results from FIG. 17 indicated other mechanisms may be involved, leading to the higher efficacy of JY108 in ameliorating EAE symptoms than MEDI551.

1. A method of treating an autoimmune disease in a subject, comprising:
administering an effective amount of a compound of formula (Ib), or a pharmaceutically acceptable salt thereof to said subject, 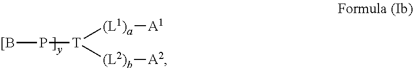
wherein:
P is a non-immunogenic polymer;
B is H or a capping group selected from C₁₋₁₀ alkyl and aryl, wherein one or more carbons of said alkyl or aryl are optionally replaced with a heteroatom;
T is a tri-functional linker having one, two, or more functional groups that, after derivatization and/or extension with a bifunctional spacer, are capable of site-specific conjugation with A¹ and A² or their derivatives, wherein the linkage between T and (L¹)ₐ and the linkage between T and (L²)_{b} can be same or different;
each of L¹ and L² is independently a bifunctional linker;
each of a and b is an integer selected from 1, 2, 3, 4, 5, 6, 7, 8, 9, and 10;
each of A¹ and A² is independently selected from an antibody or an antigen-binding fragment thereof, in which one of A¹ and A² recognizes and binds to the antigen CD3 and the other recognizes and binds the antigen CD19; and
y is an integer selected from 1, 2, 3, 4, 5, 6, 7, 8, 9, and 10;
or
administering an effective amount of a compound of formula (Ia), or a pharmaceutically acceptable salt thereof to the subject 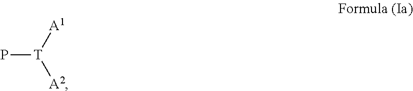
wherein
P is a non-immunogenic polymer;
T is a multifunctional small molecule linker moiety and has one, two, or more functional groups that are capable of site-specific conjugation with one, two or more the same or different polypeptides;
Each of A¹ and A² is independently selected from an antibody or an antigen-binding fragment thereof, in which one of A¹ and A² recognizes and binds to the antigen CD3 and the other recognizes and binds the antigen CD19.

2. (canceled)

3. The method of claim 1, wherein the autoimmune disease is selected from the group consisting of multiple sclerosis (MS), pemphigus, neuromyelitis optica/neuromyelitis optica-spectrum disorders (NOD/NMOD), ANCA associated vasculitis, rheumatoid arthritis (RA), Crohn's disease, Inflammatory bowel disease (IBD) and systemic lupus erythematosus (SLE), asthma, psoriasis, atopic dermatitis, erythematosus, and type 1 diabetes.

4. The method of claim 27, wherein the autoimmune disease is resistant or refractory to a conventional small molecular drug therapy, or to a conventional protein drug therapy, or to a B cell depletion therapy.

5. (canceled)

6. (canceled)

7. The method of claim 1, wherein A¹ and A² are each independently selected from a Fab, a single chain antibody, and a single domain antibody (nanobody).

8. The method of claim 1, wherein the linkage between T and A¹, the linkage between T and A², the linkage between T and (L¹)ₐ, the linkage between T and (L²)ₐ, the linkage between (L¹)ₐ and A¹, the linkage between (L²)_{b} and A², the linkage within (L¹)ₐ and the linkage within (L²)_{b} are each independently derived from functional groups selected from the group consisting of alkyl halide, acid halide, aldehyde, ketone, ester, anhydride, carboxylic acid, amide, amine, hydrazide, alkylhydrazines, hydroxy, epoxide, thiol, maleimide, 2-pyridyldithio varian, aromatic or vinyl sulfone, acrylate, bromo or iodo acetamide, azide, alkene, alkyne, dibenzocyclooctyl (DBCO), 2-amino-benzaldehyde or 2-amino-acetophenone group, hydrazide, oxime, potassium acyltrifluoroborate, O-carbamoylhydroxylamine, trans-cyclooctene, tetrazine and triarylphosphine.

9. The method of claim 1, wherein L¹ and L² each comprises a spacer independently selected from the group consisting of —(CH₂)ₘXY(CH₂)ₙ—, —X(CH₂)ₘO(CH₂CH₂O)ₚ(CH₂)ₙY—, —(CH₂)ₘX—Y(CH₂)ₙ—, —(CH₂)ₘheterocyclyl-, —(CH₂)ₘX—, —X(CH₂)ₘY—, and an amino acid or a peptide having 2 to 50 amino acid residues; wherein m, n, and p in each instance are independently an integer ranging from 0 to 25; X and Y in each instance are independently selected from the group consisting of C(═O), CR¹R², NR₃, S, O, or Null, wherein R₁ and R₂ independently represent hydrogen, C₁₋₁₀ alkyl or (CH₂)₁₋₁₀C(═O), R₃ is H or a C₁₋₁₀ alkyl, and wherein the heterocyclyl is derived from an maleimido, strained alkenes and alkynes, azide or a tetrazolyl moiety.

10. (canceled)

11. The method of claim 1, wherein P comprises polyethylene glycol (PEG) and B is methyl or a C₁₋₁₀ alkyl, and the linkage of T to P is non-cleavable or cleavable.

12. (canceled)

13. The method of claim 1, wherein P comprises a linear PEG or a branched PEG, and wherein a molecular weight of PEG ranges from 3000 Da to 80000 Da.

14. (canceled)

15. (canceled)

16. The method of claim 1, wherein the linkage of T to P is selected from the group consisting of amide, ester, carbamate, carbonate, imide, imine, hydrazones, sulfone, ether, thioether, thioester and disulfide.

17. The method of claim 1, wherein T is derived from a natural or unnatural amino acid selected from the group consisting of cysteine, lysine, asparagine, aspartic, glutamic acid, glutamine, histidine, serine, threonine, tryptophan, tyrosine or genetically-encoded alkene lysine (such as N6-(hex-5-enoyl)-L-lysine), 2-Amino-8-oxononanoic acid, m- or p-acetyl-phenylalanine, amino acid bearing a β-diketone side chain (such as 2-amino-3-(4-(3-oxobutanoyl)phenyl)propanoic acid), (S)-2-amino-6-(((1R,2R)-2-azidocyclopentyloxy)carbonylamino) hexanoic acid, azidohomoalanine, pyrrolysine analogue N6-((prop-2-yn-1-yloxy)carbonyl)-L-lysine, (S)-2-Amino-6-pent-4-ynamidohexanoic acid, (S)-2-Amino-6-((prop-2-ynyloxy)carbonylamino)hexanoic acid, (S)-2-Amino-6-((2-azidoethoxy)carbonylamino)hexanoic acid, p-azidophenylalanine, para-azidophenylalanine, Nε-Acryloyl-1-lysine, Nε-5-norbornene-2-yloxycarbonyl-1-lysine, N-ε-(Cyclooct-2-yn-1-yloxy)carbonyl)-L-lysine, N-ε-(2-(Cyclooct-2-yn-1-yloxy)ethyl) carbonyl-L-lysine, and genetically encoded tetrazine amino acid (such as 4-(6-methyl-s-tetrazin-3-yl)aminophenylalanine).

18. (canceled)

19. The method of claim 1, wherein P is derived from a PEG having a terminal maleimide or 2-pyridyldithio variant or aromatic sulfone or vinyl sulfone or azide or dibenzocyclooctyl (DECO) or oxime or trans-cyclooctene, T is derived from a natural or unnatural amino acid, and wherein (L¹)ₐ-T-(L²)_{b} is a peptide having 3-100 natural or unnatural amino acid residues.

20. The method of claim 19, wherein P is derived from a PEG having a terminal maleimide, T is derived from cysteine, and the linkage between P and T is a thioether or disulfide, and wherein (L¹)ₐ-T-(L²)_{b} is a peptide having 3-100 amino acid residues.

21. (canceled)

22. The method of claim 1, wherein the compound is selected from the group consisting of 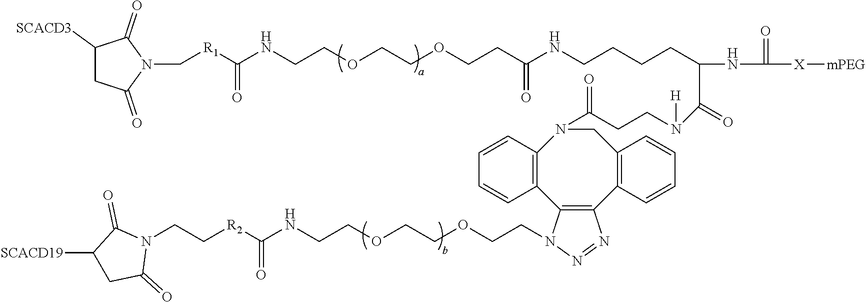
wherein the mPEG has a molecular weight of 10000 to 80000 Da;
each of a and b is independently an integer selected from 1 to 20;
X is selected from C, N, O;
each of R₁ and R₂ is independently selected from C₁₋₁₀ alkane or cyclohexane.

23. The method of claim 1, wherein the compound is administered in amounts of from about 0.05 to about 50 mg/kg/dose or from about 0.25 to about 10 mg/kg/dose.

24. (canceled)

25. The method of claim 1, wherein the compound is administered once to eight times every 4-8 weeks for each treatment cycle, or once to four times in 4-8 weeks, followed by one week rest period for each cycle until desired results are demonstrated.

26. The method of claim 1, wherein the compound is administered in combination with a second agent simultaneously or sequentially.

27. The method of claim 1, wherein the autoimmune disease is a resistant or refractory autoimmune disease.

28. (canceled)

29. The method of claim 4, wherein the autoimmune disease is resistant or refractory to a therapy with Tecfidera, Gilenya, Tysabri, Aubagio, or Mavenclad;
or to a therapy with Copaxone, IFN-β-1a, IFN-β-1b, anti-CD52 antibodies (Alemtuzumab, Alemtuzumab), or Natalizumab;
or to a therapy with anti-CD19 agent (Inebilizumab, Obexelimab) or anti-CD20 agent (Rituximab, ocrelizumab, ofatumumab).

30. The method of claim 22, wherein the compound has the following structure: 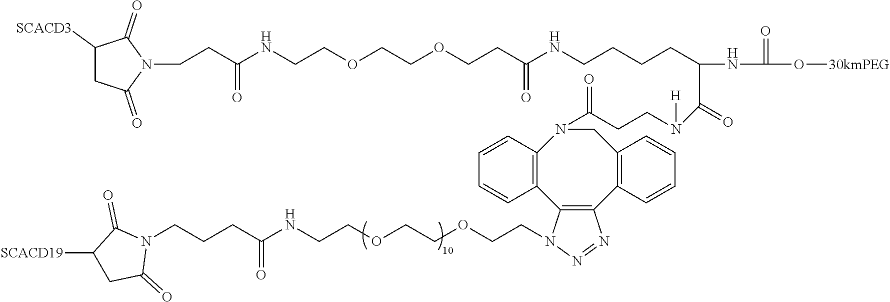
wherein, the SCACD19 has the amino acid sequence as set forth in SEQ ID NO. 1 and the SCACD3 has the amino acid sequence as set forth in SEQ ID NO: 2.

31. The method of claim 26, wherein the second agent is Tecfidera, Gilenya, Tysabri, Aubagio, Mavenclad, Copaxone, IFN-β-1a, IFN-β-1b, anti-CD52 antibody (Alemtuzumab, Alemtuzumab), Natalizumab, anti-CD19 agent (Inebilizumab, Obexelimab), or anti-CD20 agent (Rituximab, ocrelizumab, ofatumumab).
